(19) United States
(12) Patent Application Publication
(10) Pub. No.: US 2026/0069268 A1
(43) Pub. Date: Mar. 12, 2026

(54) SMALL BONE FIXATION SYSTEMS AND TECHNIQUES FOR COMBINED STAPLE AND SCREW FIXATION

(71) Applicant: Treace Medical Concepts, Inc., Ponte Vedra, FL (US)

(72) Inventors: Adriaan Kuyler, Ponte Vedra, FL (US); Sean F. Scanlan, Jacksonville, FL (US); Adam Hausman, Jacksonville, FL (US); Jason May, St. Johns, FL (US); Patrick Murray, Ponte Vedra, FL (US)

(21) Appl. No.: 19/325,233

(22) Filed: Sep. 10, 2025

Related U.S. Application Data
(60) Provisional application No. 63/693,159, filed on Sep. 10, 2024.

(51) Int. Cl. A61B 17/064; A61B 17/00; A61B 17/17; A61B 17/56; A61B 17/86; A61B 17/88; A61B 17/90

(52) U.S. Cl. CPC A61B 17/0642; A61B 17/1775; A61B 17/86; A61B 17/8872; A61B 17/90; A61B 2017/00477; A61B 2017/0641; A61B 2017/565

(57) ABSTRACT

Devices, systems, and techniques can be used to facilitate insertion of staple implants that include a combination of legs (e.g., tines) and fixation holes that receive screws therethrough. In some examples, an implant insertion system includes a guide body operatively connected to connector that joins coupling shafts that energize the staple during insertion. The guide body can include a first guide opening configured to guide drilling of a first hole into a first bone aligned with the first fixation aperture of the staple and/or insertion of a first screw into the first bone through the first fixation aperture. The guide body can also include a second guide opening configured to guide drilling of a second hole into a second bone aligned with the second fixation aperture and/or insertion of a second screw into the second bone through the second fixation aperture.

### CROSS-REFERENCE

This application claims the benefit of U.S. Provisional Patent Application No. 63/693,159, filed Sep. 10, 2024, the entire contents of which is incorporated herein by reference.

### TECHNICAL FIELD

This disclosure generally relate to devices, systems, and techniques for small bone anatomy in the foot.

### BACKGROUND

Bones within the human body, such as bones in the foot, may be anatomically misaligned. For example, one common type of bone deformity is hallux valgus, which is a progressive foot deformity in which the first metatarsophalangeal joint is affected and is often accompanied by significant functional disability and foot pain. The metatarsophalangeal joint is laterally deviated, resulting in an abduction of the first metatarsal while the phalanges adduct. This often leads to development of soft tissue and a bony prominence on the medial side of the foot, which is called a bunion.

Surgical intervention may be used to correct a bunion deformity. A variety of different surgical procedures exist to correct bunion deformities and may involve removing the abnormal bony enlargement on the first metatarsal and/or realigning the first metatarsal relative to the adjacent metatarsal. In some procedures, an implant can be used to fixate a position of the metatarsal after realignment. The implant can be applied between the metatarsal and opposed cuneiform, across the tarsometatarsal joint. Such an implant can hold the metatarsal in a realigned position while bone grows to form a fused connection between the metatarsal and opposed cuneiform.

### SUMMARY

This disclosure is generally directed to devices, systems, and techniques for facilitating insertion of staple implants that include a combination of legs (e.g., tines) and fixation holes that receive screws therethrough. For example, the disclosed devices, systems, and techniques can be used to facilitate installation of a staple that includes a first leg and a first fixation aperture on a first side of the staple, a second leg and a second fixation aperture on a second side of the staple, and a bridge connecting the first side of the staple to the second side of the staple. The staple can be elastically deformed for insertion using a first coupling shaft connected to a first handling coupling through the top surface of the staple and a second coupling shaft connected to a second handling coupling through the top surface of the staple. A connector can join the first coupling shaft and the second coupling shaft together.

In some examples as described herein, an implant insertion system includes a guide body operatively connected to the connector. The guide body can include a first guide opening configured to guide drilling of a first hole into a first bone aligned with the first fixation aperture of the staple and/or insertion of a first screw into the first bone through the first fixation aperture. The guide body can also include a second guide opening configured to guide drilling of a second hole into a second bone aligned with the second fixation aperture and/or insertion of a second screw into the second bone through the second fixation aperture.

In some applications, a clinician can use a separate drill guide to pre-drill holes in the bones where the legs of the staple are to be placed. For example, the clinician can prepare first, second, third, and fourth staple leg openings by drilling holes through corresponding first, second, third, and fourth staple leg guide openings of a staple leg drill guide. The clinician can then insert first, second, third, and fourth legs of the staple into the holes with coupling shafts attached to the staple energized and joined by a connector. The clinician can then use a guide body operatively connected to the connector to drill first and second holes aligned with first and second fixation apertures of the staple. In some examples, the clinician guides a drill bit through guide tubes inserted into guide body openings of the guide body and then removes the guide tubes from the guide body openings. In either case, the clinician can then guide screws through the guide openings defined by the guide body and into the fixation apertures of staple. In this way, the system can facilitate accurate drilling of openings and placement of screws through staple fixation apertures in small bone anatomy with limited access space.

The details of one or more examples are set forth in the accompanying drawings and the description below. Other features, objects, and advantages will be apparent from the description and drawings, and from the claims.

### BRIEF DESCRIPTION OF DRAWINGS

FIGS. 1A and 1B are front views of a foot showing a normal first metatarsal position and an example frontal plane rotational misalignment position, respectively.

FIGS. 2A and 2B are top views of a foot showing a normal first metatarsal position and an example transverse plane misalignment position, respectively.

FIGS. 3A and 3B are side views of a foot showing a normal first metatarsal position and an example sagittal plane misalignment position, respectively.

FIGS. 4A and 4B illustrate an orthopedic implant in the form of a staple. FIG. 5A is a perspective view of this embodiment of the staple, and FIG. 5B is a side elevational view of a longitudinal cross-section of the staple shown at FIG. 5A.

FIG. 5 is a perspective view of another example configuration of a staple.

FIGS. 6A and 6B illustrate an additional embodiment of an orthopedic implant in the form of a staple that includes fixation apertures. FIG. 6A is a perspective view of this embodiment of the staple showing the fixation apertures, and FIG. 6B is a perspective view of this embodiment of the staple showing fixation members placed at the fixation apertures.

FIGS. 7A and 7B illustrate a further embodiment of an orthopedic implant in the form of a staple that includes fixation apertures as well as certain exemplary anatomical contouring. FIG. 7A is a side elevational view of this embodiment of the staple showing exemplary anatomical contouring, and FIG. 7B is a side elevational view showing such staple embodiments with exemplary anatomical contouring placed across a space (e.g., a joint space) separating first and second bones.

FIG. 8 is a side view of an additional embodiment of an orthopedic implant in the form of a staple that includes one or more fixation aperture.

FIGS. 9A-9C illustrate an embodiment of an inserter. FIG. 9A shows disassembled components of this inserter, FIG. 9B shows assembled components of this inserter, and FIG. 9C shows this inserter operatively connected to a staple with a load force removed such that the staple is in its biased, compression state.

FIGS. 10A-10E illustrate an example staple leg drill guide having one or more guide openings, each opening being configured to guide drilling of a hole into an underlying bone at a location where a leg of a staple is to be inserted.

FIG. 11 illustrates an example staple implant insertion device.

FIG. 12 is a side view of the example staple implant insertion device showing an example configuration of the device where guide lumens are defined by closed sidewalls.

FIG. 13 is a side view of the example staple implant insertion device showing an example configuration of guide tubes with a guide body.

FIGS. 14A-14D illustrate a variety of different example guide tube and guide lumen configurations that can be used on an example staple implant insertion device.

FIGS. 15A and 15B are different perspective views of example configurations of a staple implant insertion device.

FIG. 16A is an alternative configuration of an example drill guide.

FIG. 16B illustrates an example configuration of a staple implant insertion device that can be used after forming openings in bones using the example drill guide of FIG. 16A.

FIGS. 17A-17C are different views of an example configuration of a staple implant insertion device in which the staple implant insertion device includes a converging receiving cavity.

FIGS. 18A-18D illustrate different example lock features that can be used on a staple implant insertion device according to the disclosure.

FIGS. 19A-19C are side view illustrations of an example configuration of a staple implant insertion device showing example procedure steps for engaging the staple implant insertion device with a first coupling shaft and a second coupling shaft attached to a staple.

FIGS. 20A and 20B are sectional side views of an example configuration of a staple implant insertion device showing a first coupling shaft and a second coupling shaft attached to a staple and being inserted into an example receiving opening of the device.

FIGS. 21A-21C are perspective views of example procedural steps that can be performed with an example staple implant insertion device after inserting the legs of the staple into bone portions.

Like reference characters are used in the following description and in the drawings to indicate like elements.

### DETAILED DESCRIPTION

This disclosure generally relates to devices, systems (e.g., kits), and techniques for fixating one or more bones using one or more guide bodies to guide drilling and/or screw insertion through a fixation device underlying bone portions. In some examples, an implant is in the form of a staple that includes a first leg and a first fixation aperture on a first side of the staple, a second leg and a second fixation aperture on a second side of the staple, and a bridge connecting the first side of the staple to the second side of the staple. The implant insertion system includes a first coupling shaft configured to connect to a first handling coupling on the first side of the staple, a second coupling shaft configured to connect to a second handling coupling on the second side of the staple, and a connector that is configured to join the first coupling shaft and the second coupling shaft to facilitate insertion of the staple legs into underlying bone portions.

As will be described, an example implant insertion system can also include a guide body operatively connected to the connector joining the coupling shafts. The guide body can define a first guide opening configured to guide drilling of a first hole into a first bone aligned with the first fixation aperture and/or insertion of a first screw into the first bone through the first fixation aperture. The guide body may additionally or alternatively include a second guide opening configured to guide drilling of a second hole into the second bone aligned with the second fixation aperture and/or insertion of a second screw into the second bone through the second fixation aperture. In use, a clinician may use a separate staple leg drill guide to drill a first staple leg hole into the first bone and a second staple leg hole into the second bone. The clinician can then insert the first leg of the staple into the first staple leg hole and the second leg of the staple into the second staple leg hole, with the staple legs being biased away from each other via positioning of the first coupling shaft relative to the second coupling shaft and joined together with the connector. With the first leg of the staple inserted into the first staple leg hole and the second leg of the staple inserted into the second staple leg hole, the clinician can use the guide body operatively connected to the connector to guide drilling of a hole and/or insertion of a screw through the first and/or second fixation aperture of the staple.

In some examples, devices, systems, and techniques according to the disclosure can be used to fixate a repositioned bone, or bones, during a surgical procedure, such as a metatarsal realignment and fusion procedure and/or a phalanx realignment and fusion procedure. In exemplary applications, the devices, systems, and techniques can be used during a surgical procedure performed on one or more bones, such as a bone alignment, osteotomy, fusion procedure, fracture repair, and/or other procedures where one or more bones are to be set in a desired position. Such a procedure can be performed, for example, on bones (e.g., adjacent bones separated by a joint or different portions of a single bone) in the foot or hand, where bones are relatively small compared to bones in other parts of the human anatomy. In one example, a procedure utilizing devices and/or techniques of the disclosure can be performed to correct an alignment between a metatarsal (e.g. a first metatarsal) and a cuneiform (e.g., a medial cuneiform), such as a bunion correction. An example of such a procedure is a lapidus procedure. In another example, the devices, systems, and/or techniques can be utilized when modifying a position of one portion of a bone relative to another portion of the same bone. An example of such a procedure is an osteotomy procedure (e.g., metatarsal osteotomy procedure) in which the bone is cut into at least two different bones and one portion (e.g., a distal portion) is realigned relative to another bone portion (e.g., a proximal portion) of the same bone.

In various examples, the devices, systems, and/or techniques of the disclosure may be utilized on comparatively small bones in the foot such as a metatarsal (e.g., first, second, third, fourth, or fifth metatarsal), a cuneiform (e.g., medial, intermediate, lateral), a cuboid, a phalanx (e.g., proximal, intermediate, distal), and/or combinations thereof. The bones may be separated from each other by a tarsometatarsal (“TMT”) joint, a metatarsophalangeal (“MTP”) joint, or other joint.

In some examples, the present disclosure is directed to devices and techniques for preparing the metatarsophalangeal (“MTP”) joint for fusion and realigning the metatarsal and phalanx separated by the joint. While a technique according to the disclosure can be performed on any MTP joint where a metatarsal is joined to an opposing proximal phalanx, in some implementations, the technique is performed on the first MTP joint where the first metatarsal joins the first proximal phalanx. During the procedure, a metatarsal may be separated from an opposing proximal phalanx at the MTP joint and both the metatarsal and opposing phalanx repositioned within one or more planes. After suitably repositioning the metatarsal and opposed proximal phalanx, the bone portions may be fixed to each using one or more fixation devices crossing the MTP joint, as described herein. The end faces of the metatarsal and opposed proximal phalanx can be prepared prior to fixation, e.g., to promote realignment and/or subsequent fusion of the bones to each other.

Preparation and fusion of a metatarsal and phalanx may be performed according to the disclosure for a variety of clinical reasons and indications. Preparation and fusion of a metatarsal and proximal phalanx at the MTP joint may be performed to treat hallux rigidus, hallux valgus, and/or other bone and/or joint conditions.

While techniques and devices are described herein particularly in connection with the first metatarsal and first proximal phalanx of the foot, the techniques and devices may be used on other adjacent bones separated by a joint in the hand or foot. For example, the techniques and devices may be performed on a different metatarsal (e.g., second, third, fourth, or fifth metatarsal) and its opposed proximal phalanx. Additional details on systems, devices, and techniques that can be used in conjunction with teachings of the present disclosure can be found in U.S. patent application Ser. No. 18/467,688, filed Sep. 14, 2023, and titled “BONE FIXATION TECHNIQUES AND IMPLANTS” and U.S. patent application Ser. No. 18/437,056, filed Feb. 8, 2024, and titled “SMALL BONE FIXATION SYSTEMS AND TECHNIQUES WITH GUIDE SLEEVE,” the entire contents of each of which are incorporated herein by reference.

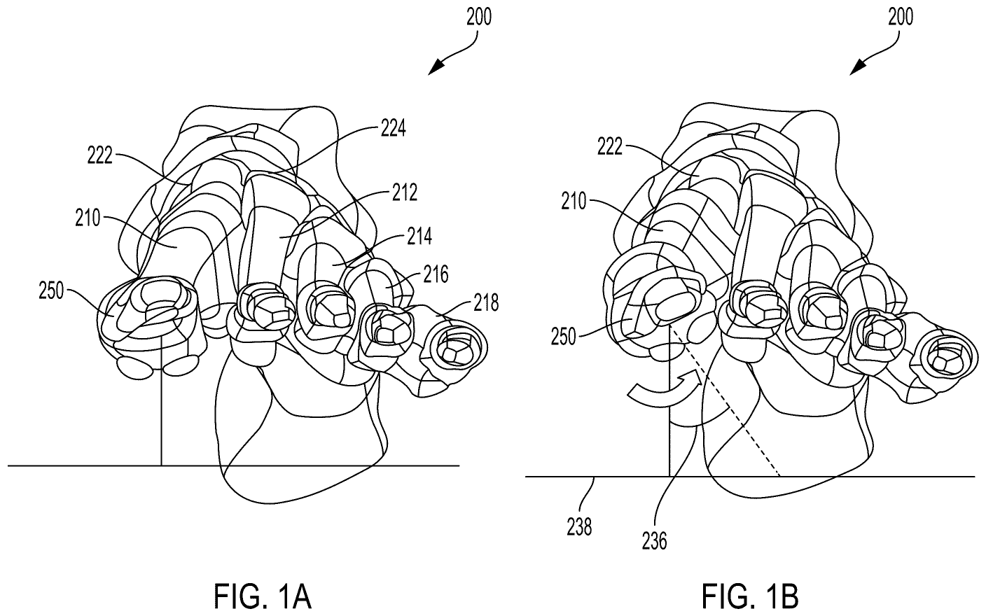

To further understand example techniques of the disclosure, the anatomy of the foot will first be described with respect to FIGS. 1-3 along with example misalignments that may occur and be corrected according to the present disclosure. As noted, a bone misalignment and/or MTP joint pain may be caused by hallux valgus (bunion), hallux rigidus, a natural growth deformity, and/or other condition. The condition may present with a misalignment of one or more bones in the foot. Alternatively, the condition may present with evidence of arthritis at the MTP joint without visible misalignment of the bone forming the joint.

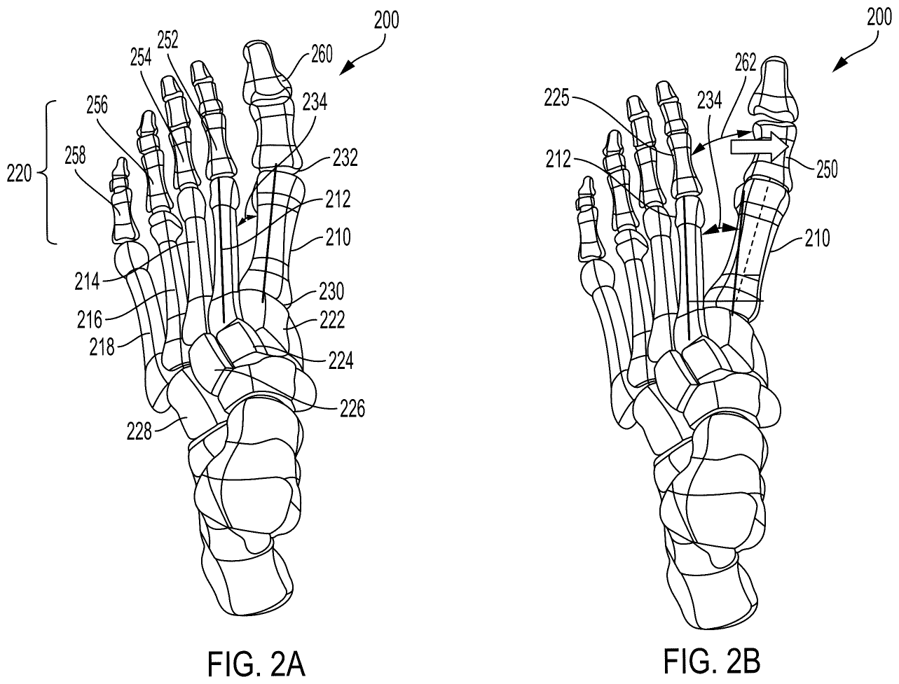

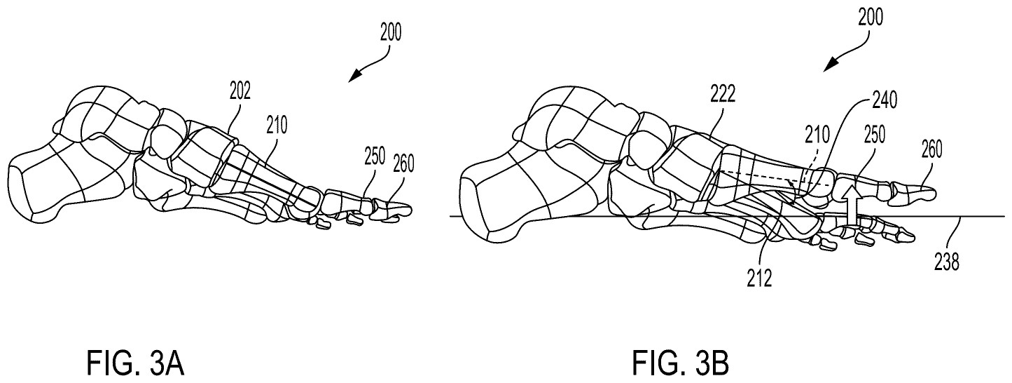

FIGS. 1A and 1B are front views of foot 200 showing a normal first metatarsal position and an example frontal plane rotational misalignment position, respectively. FIGS. 2A and 2B are top views of foot 200 showing a normal first metatarsal position and an example transverse plane misalignment position, respectively. FIGS. 3A and 3B are side views of foot 200 showing a normal first metatarsal position and an example sagittal plane misalignment position, respectively. While FIGS. 1B, 2B, and 3B show each respective planar misalignment in isolation, in practice, a metatarsal may be misaligned in any two of the three planes or even all three planes. Accordingly, it should be appreciated that the depiction of a single plane misalignment in each of FIGS. 1B, 2B, and 3B is for purposes of illustration and a metatarsal may be misaligned in multiple planes that is desirably corrected. Further, a bone condition treated according to the disclosure may not present any of the example misalignments described with respect to FIGS. 1B, 2B, and 3B, and it should be appreciated that the disclosure is not limited in this respect.

With reference to FIGS. 1A and 2A, foot 200 is composed of multiple bones including a first metatarsal 210, a second metatarsal 212, a third metatarsal 214, a fourth metatarsal 216, and a fifth metatarsal 218. The metatarsals are connected distally to phalanges 220 and, more particularly, each to a respective proximal phalanx. In particular, the first metatarsal 210 is connected distally to first proximal phalanx 250, the second metatarsal 212 is connected distally to second proximal phalanx 252, the third metatarsal 214 is connected distally to third proximal phalanx 254, the fourth metatarsal 216 is connected distally to fourth proximal phalanx 256, and the fifth metatarsal 218 is connected distally to fifth proximal phalanx 258. The joint 232 between a metatarsal and a corresponding opposed proximal phalanx is referred to as a metatarsophalangeal (“MTP”) joint. The first MTP joint is labeled as joint 232 in FIG. 2A, although second, third, fourth, and fifth MTP joints are also illustrated in series adjacent to the first MTP joint.

The first metatarsal 210 is connected proximally to a medial cuneiform 222, while the second metatarsal 212 is connected proximally to an intermediate cuneiform 224 and the third metatarsal is connected proximally to lateral cuneiform 226. The fourth and fifth metatarsals 216, 218 are connected proximally to the cuboid bone 228. The joint 230 between a metatarsal and respective cuneiform (e.g., first metatarsal 210 and medial cuneiform 222) is referred to as the tarsometatarsal (“TMT”) joint. The angle 234 between adjacent metatarsals (e.g., first metatarsal 210 and second metatarsal 212) is referred to as the intermetatarsal angle (“IMA”).

As noted, FIG. 1A is a frontal plane view of foot 200 showing a typical position for first metatarsal 210. The frontal plane, which is also known as the coronal plane, is generally considered any vertical plane that divides the body into anterior and posterior sections. On foot 200, the frontal plane is a plane that extends vertically and is perpendicular to an axis extending proximally to distally along the length of the foot. FIG. 1A shows first metatarsal 210 in a typical rotational position in the frontal plane. FIG. 1B shows first metatarsal 210 with a frontal plane rotational deformity characterized by a rotational angle 236 relative to ground, as indicated by line 238.

FIG. 2A is a top view of foot 200 showing a typical position of first metatarsal 210 in the transverse plane. The transverse plane, which is also known as the horizontal plane, axial plane, or transaxial plane, is considered any plane that divides the body into superior and inferior parts. On foot 200, the transverse plane is a plane that extends horizontally and is perpendicular to an axis extending dorsally to plantarly (top to bottom) across the foot. FIG. 2A shows first metatarsal 210 with a typical IMA 234 in the transverse plane. FIG. 2B shows first metatarsal 210 with a transverse plane rotational deformity characterized by a greater IMA caused by the distal end of first metatarsal 210 being pivoted medially relative to the second metatarsal 212.

FIG. 3A is a side view of foot 200 showing a typical position of first metatarsal 210 in the sagittal plane. The sagittal plane is a plane parallel to the sagittal suture which divides the body into right and left halves. On foot 200, the sagittal plane is a plane that extends vertically and intersects an axis extending proximally to distally along the length of the foot. FIG. 3A shows first metatarsal 210 with a typical rotational position in the sagittal plane. FIG. 3B shows first metatarsal 210 with a sagittal plane rotational deformity characterized by a rotational angle 240 relative to ground, as indicated by line 238.

Bone positioning techniques and instruments can be useful to correct a misalignment of one or more bones, such as the metatarsal and opposed proximal phalanx, and/or promote fusion of the metatarsal and proximal phalanx across the MTP joint. In some applications, the technique involves releasing the MTP joint and preparing the end faces of the metatarsal and proximal phalanx for realignment relative to each other and/or fusion. The metatarsal undergoing the procedure may be moved in at least one plane, or at least two planes, such as all three planes, to provide a moved position for fusing with the proximal phalanx. The proximal phalanx undergoing the procedure may additionally or alternatively be moved in at least one plane, such as two or all three planes, relative to the metatarsal and/or an adjacent proximal phalanx. Once the metatarsal and proximal phalanx are appropriately repositioned, the metatarsal and proximal phalanx can be fixated to hold and maintain their relative positions to each other, e.g., and to promote fusion between the bones.

To prepare the end face of the first metatarsal 210 and/or the end face of the first proximal phalanx 250, a tissue removing instrument can be applied to the end face. Example tissue removing instruments that can be used include, but are not limited to, a saw, a rotary bur, a rongeur, a reamer, an osteotome, and the like. The tissue removing instrument can be applied to the end face of the bone being prepared to remove cartilage and/or bone. For example, the tissue removing instrument may be applied to the end face to remove cartilage (e.g., all cartilage) down to subchondral bone. Additionally or alternatively, the tissue removing instrument may be applied to cut, fenestrate, morselize, and/or otherwise reshape the end face of the bone and/or form a bleeding bone face to promote fusion. In instances where a cutting operation is performed to remove an end portion of first metatarsal 210 and/or first proximal phalanx 250, the cutting may be performed freehand or with the aid of cutting guide having a guide surface positionable over the portion of bone to be cut. When using a cut guide, a cutting instrument can be inserted against the guide surface (e.g., between a slot define between two guide surfaces) to guide the cutting instrument for bone removal.

Before or after preparing one or both end faces of first metatarsal 210 and/or first proximal phalanx 250, the first metatarsal may be moved (e.g., relative to an adjacent metatarsal, such as a second metatarsal 212, and/or the first proximal phalanx 250) to establish a moved position of the metatarsal. Additionally or alternatively, first proximal phalanx 250 can be moved (relative to first metatarsal 210) to establish a moved position of the phalanx. In different examples, movement of the first metatarsal and/or proximal phalanx may be performed freehand (e.g., without the aid of a bone positioning device) and/or using instrumentation (e.g., a bone positioning device) to help facilitate repeatable repositioning outcomes.

First metatarsal 210 and/or first proximal phalanx 250 can be moved in at least one plane (e.g., relative to an opposed bone and/or an adjacent bone), such as at least two planes, or all three planes. The at least one plane can be the transverse plane, the frontal plane, and/or the sagittal plane. The clinician can move the bone in one plane at a time, resulting in multiple different movements to achieve a multi-planar movement. Additionally or alternatively, the clinician may perform a single repositioning of the bone in multiple planes.

In some examples, after preparing the end faces of first metatarsal 210 and first proximal phalanx 250 and moving one or both bones, the clinician may bring the prepared end face of the proximal phalanx in apposition to the prepared end face of the metatarsal (e.g., compressing the end faces together) to facilitate fusion prior to applying one or more permanent fixation devices. In some examples, an axial wire or pin may be placed to extend proximally out of the end face of the proximal phalanx. When used, the proximal phalanx may slide along the wire or pin when bringing the bone faces into apposition and/or during compression.

During a surgical technique, a clinician can apply at least one bone fixation device across or through the MTP joint separating the metatarsal from the opposed proximal phalanx. Any one or more bone fixation devices can be used including, but not limited to, a compressing bone screw, a bone plate, a bone staple, an external fixator, and/or an intramedullary implant. The bone fixation device may be secured on one side to the metatarsal, bridge the MTP joint, and be secured on an opposite side to the proximal phalanx. In some examples, the clinician uses one or more staples to fixate the metatarsal to the opposed proximal phalanx across the MTP joint. In some examples, the staple includes one or more staple legs along with one or more fixation apertures receiving one or more corresponding screws therethrough.

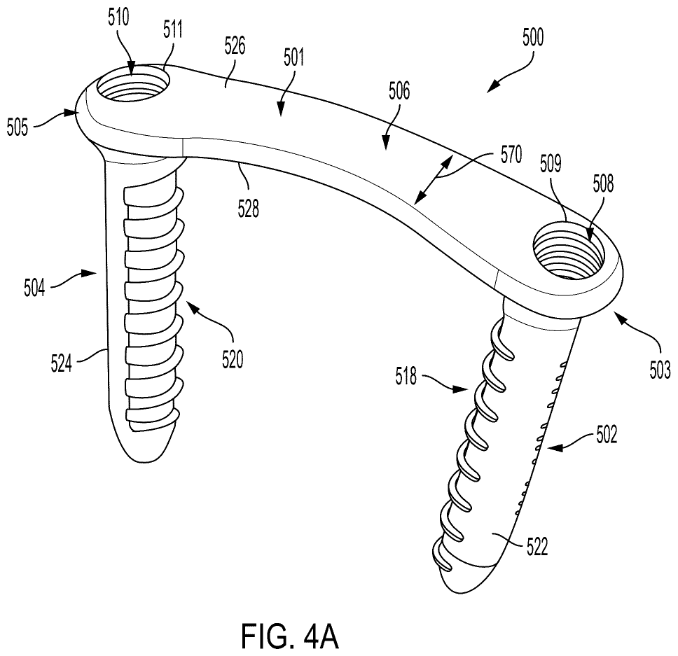

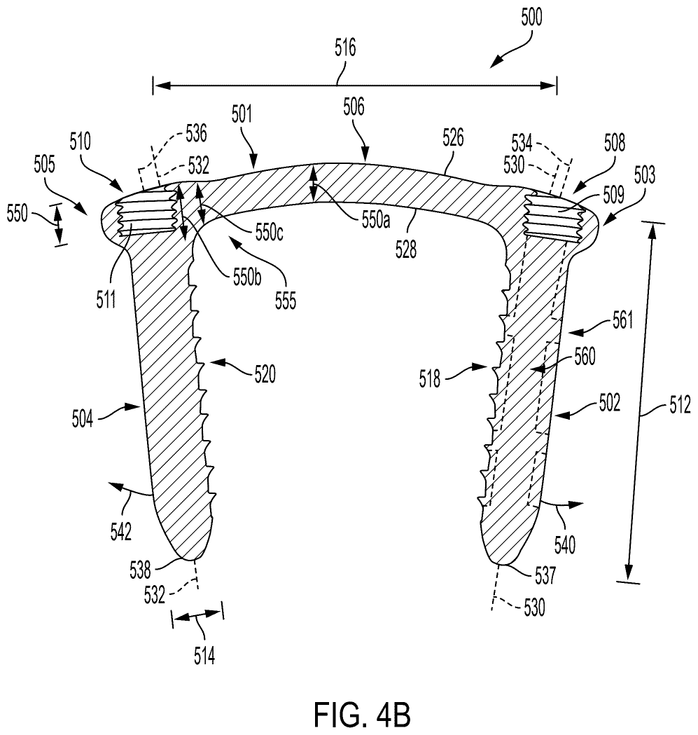

FIGS. 4-8 illustrate various embodiments of a staple as one exemplary type of implant that can be positioned and used to fixate bones for fusion.

FIGS. 4A and 4B illustrate an embodiment of a staple 500. FIG. 4A is a perspective view of the staple 500, and FIG. 4B is a side elevational view of a longitudinal cross-section of the staple 500. The staple 500 can be configured to apply a compression force at the bones and across the space (e.g., joint) between the bones for use in fixating and fusing bones.

The staple 500 can include a staple body 501 having a first leg 502, a second leg 504, and a bridge 506. For the illustrated embodiment, the staple 500 includes the first leg 502 at a first side 503 of the staple 500 and the second leg 504 at a second side 505 of the staple 500. In this example, the first side 503 is opposite the second side 505. The bridge 506 can connect the first leg 502 and the second leg 504.

The legs 502, 504 of the staple 500 can be configured for positioning in bones, such as one or more relatively small bones of the foot. For example, a length 512 of each of the legs 502, 504 can range from 8 mm to 25 mm, which can provide sufficient length to robustly anchor within a bone of the foot, such as a metatarsal (e.g., first metatarsal) and/or phalanx (e.g., proximal phalanx). A width 514 of each of the legs can range from 2 mm to 3 mm, which likewise can provide sufficient length to robustly anchor within a bone of the foot. A bridge length 516 of the bridge 506 can range from 12 mm to 20 mm, which can be sufficient to allow for positioning the bridge across a space (e.g. joint) between bones in the foot while maintaining the legs 502, 504 at such bones separated by the space. A staple according to the disclosure can be configured with dimensions other than the foregoing examples, and the disclosure is not limited in this respect.

As an example shown at FIG. 4B, the bridge length 516 can be as measured from a central longitudinal axis of one leg closest to one side of the bridge 506 to a central longitudinal axis of another leg closest to an opposite side of the bridge 506. The bridge 506 can also define a bridge width 570. In some examples, the bridge width 570 of the staple 500 is constant over the entire width of the staple. In other examples, the bridge width 570 of the staple 500 varies over the width of the staple. For example, the bridge width 570 can be substantially constant over a central region along the bridge length 516, and the bridge width 570 can increase moving along the bridge length 516 from the central region toward each of the first leg 502 and the second leg 504 (e.g., such that the greatest bridge width 570 is at or adjacent the first and second legs 502, 504). As one specific such example, the bridge width 570 can define a generally hourglass profile with the narrow, center of the hourglass at a central region along the bridge length 516 and the increasing width end portions of the hourglass at opposite ends of the bridge length 516.

The first leg 502 can include a first set of teeth 518 at a perimeter 522 of the first leg 502, and the second leg 504 can include a second set of teeth 520 at a perimeter 524 of the second leg 504. The teeth 518, 520 can extend out from the respective leg 502, 504 and be configured to provide an anchoring mechanism for maintaining the respective leg 502, 504 within the respective bone at which the respective leg 502, 504 is placed. As illustrated here, the first set of teeth 518 can extend partially around the perimeter 522 of the first leg 502, and the second set of teeth 520 can extend partially around the perimeter 524 of the second leg 504. For example, the first set of teeth 518 can extend around a portion of the perimeter 522 of the first leg 502 facing the bridge 506, and the second set of teeth 520 can extend around a portion of the perimeter 525 of the second leg 504 facing the bridge 506. As one specific such example, the first set of teeth 518 can extend around approximately one hundred and eighty degrees of the perimeter 522 of the first leg 502 nearest the bridge 506, and the second set of teeth 520 can extend around approximately one hundred and cighty degrees of the perimeter 524 of the second leg 504 nearest the bridge 506.

The staple 500 can further include a first handling coupling 508 and a second handling coupling 510 defined by the staple body 501. For the illustrated embodiment, the staple 500 includes the first handling coupling 508 at the first side 503 of the staple 500 and the second handling coupling 510 at the second side 505 of the staple 500. The first handling coupling 508 can include a first handling coupling receptacle 509 extending from a top surface 526 of the staple body 501 of the staple 500 toward (e.g., to) a bottom surface 528 of the staple body 501 of the staple 500. As one such specific example, the first handling coupling receptacle 509 can extend from the top surface 526 down a portion, but less than all of, of the length 512 of the first leg 502. The second handling coupling 510 can include a second handling coupling receptacle 511 extending from the top surface 526 of the staple body 501 of the staple 500 toward (e.g., to) the bottom surface 528 of the staple body 501 of the staple 500. As one such specific example, the second handling coupling receptacle 511 can extend from the top surface 526 down a portion, but less than all of, of the length 512 of the second leg 504. As such, the first handling coupling 508 and first handling coupling receptacle 509 as well as the second handling coupling 510 and second handling coupling receptacle 511 can be accessible from the top surface 526 of the staple 500 which can be useful in helping to facilitate generally flush placement of the bottom surface 528 of the staple 500 against one or more bones (e.g., against each of two bones separated by a space, such as a joint).

The first handling coupling receptacle 509 of the first handling coupling 508 can be configured to couple to a first coupling shaft of an inserter, such as at a location between the top surface 526 and the bottom surface 528. The second handling coupling receptacle 511 of the second handling coupling 510 can be configured to couple to a second coupling shaft of an inserter, such as at a location between the top surface 526 and the bottom surface 528. As such, the first and second handling coupling receptacles 509, 511 can be configured to operatively couple to the respective first and second coupling shafts of the inserter such that the first and second coupling shafts of the inserter are inserted into the respective first and second handling coupling receptacles 509, 511 from the top surface 526 and maintained within the respective first and second handling coupling receptacles 509, 511 so as to not extend out from the bottom surface 528. The illustrated embodiment of the first and second handling coupling receptacles 509, 511 includes threads extending along a length of the first and second handling coupling receptacles 509, 511 between the top and bottom surfaces 526, 528, and these threads can be configured to connect to complementary threads at the respective first and second coupling shafts of the inserter. Though in other embodiments the first and second handling coupling receptacles 509, 511 and first and second coupling shafts of the inserter can include others means to facilitate an operative connection therebetween the respective components.

Depending on the application in which the staple 500 is used, the staple 500 can be configured to receive one or more solid or liquid substances after insertion of the staple into bone. As one such example, one or both of the first and second handling coupling receptacles 509, 511 can be configured to receive a filler material therein to substantially plug the first and/or second handling coupling receptacles 509, 511 at the top surface 526. This filler material can be placed in the first and/or second handling coupling receptacles 509, 511 after removing the respective first and/or second coupling shaft from the respective first and/or second handling coupling receptacles 509, 511. For instance, a biologically compatible wax or other biologically compatible filler material can be placed into the first and/or second handling coupling receptacles 509, 511 to plug the first and/or second handling coupling receptacles 509, 511 at or near the top surface 526 so as to help impede passage of biologic substances into the first and/or second handling coupling receptacles 509, 511.

As another such example, one or both of the legs 502, 504 can be configured to receive and convey a substance therethrough. For instance, a cannula 560 can be defined within one or both of the legs 502, 504, and the cannula 560 can extend along at least a portion (e.g., all) of the length 512 of the leg 502 and/or 504. When included, the cannula 560 can have an inlet, for instance at the respective handling coupling 508, 510, and the cannula 560 can have one or more outlets 561 at a location along the respective leg 502, 504 spaced apart from the respective handling coupling 508, 510. For the illustrated embodiment, the outlets 561 can be included at the respective leg 502 and/or 504 between teeth 518, 520. A medication, structural support substance, or other biologically compatible substance can be introduced into the cannula 560 at the inlet (e.g., at the respective handling coupling 508, 510) and this substance can be delivered to one or more bones, at which the staple 500 is placed, via the one or more outlets 561.

Additionally or alternatively, the staple 500 can be configured with a cannulation extending through the length of leg 502 and/or 504 for receiving corresponding wires inserted into bone to help facilitate positioning and placement of the staple into underlying bone. For example, in lieu of using an inserter having wire receiving openings to guide positioning of an implant as will be described, wires inserted into underlying bones can be aligned with cannulations extending through at least two legs of the staple. The cannulations can be aligned with the wires positioned in the bones and the staple guided along the wires.

The first leg 502 can define, and length 512 of the leg can extend along, a first leg central longitudinal axis 530, and the second leg 504 can define, and the length 512 of the leg can extend along, a second leg central longitudinal axis 532. Likewise the first handling coupling receptacle 509 can define, and extend a length from the top surface 526 toward (e.g., to) the bottom surface 528 along, a first handling coupling receptacle central longitudinal axis 534, and the second handling coupling receptacle 511 can define, and extend a length from the top surface 526 toward (e.g., to) the bottom surface 528 along, a second handling coupling receptacle central longitudinal axis 536.

As shown for the illustrated embodiment of the staple 500 at FIG. 4B, the first leg central longitudinal axis 530 can be offset from the first handling coupling receptacle central longitudinal axis 534, and the second leg central longitudinal axis 532 can be offset from the second handling coupling receptacle central longitudinal axis 536. In particular, in this illustrated embodiment of the staple 500, the first leg central longitudinal axis 530 can be closer to the bridge 506 than the first handling coupling receptacle central longitudinal axis 534, and the second leg central longitudinal axis 532 can be closer to the bridge 506 than the second handling coupling receptacle central longitudinal axis 536. This offset arrangement can be helpful to increase a cross-sectional area at an intersection of the bridge 506 and the first leg 502 and/or the second leg 504 and, thereby, help to increase the ability of the staple body 501 of the staple 500 to accommodate a load force, applied at the staple 500, in a manner that results in clastic deformation of the staple 500 as the staple 500 moves between a natural, undeformed state and a deformed insertion state upon application/removal of a load force.

The staple 500 can have a thickness 550 that can differ at different regions of the staple 500. For example, the staple 500 can have a bridge thickness 550a at the bridge 506, a leg thickness 550b at the first leg 502 and the second leg 504, and a thickness transition region 555 where the bridge 506 transitions to the respective first leg 502 and the second leg 504. As shown for the illustrated embodiment, the leg thickness 550b can be greater than the bridge thickness 550a (e.g., at a central location of the bridge along the bridge length 516), and the thickness transition region 555 can have a thickness transition region thickness 550c that is greater than the bridge thickness 550a and less than the leg thickness 550b. In particular, the thickness transition region 555 can include an increase in thickness of the staple 500 moving in a direction from the bridge 506 toward the respective leg 502, 504. In one example, the first handling coupling 508 and the first handling coupling receptacle 509 can be located at the thickness transition region 555 adjacent the first leg 502, and the second handling coupling 510 and the second handling coupling receptacle 511 can be located at the thickness transition region 555 adjacent the second leg 504. Such location of the first handling coupling 508 and the first handling coupling receptacle 509 as well as the second handling coupling 510 and the second handling coupling receptacle 511 at the increased thickness portion of the staple 500 can help to increase the strength of the staple 500 for receiving a load force.

The staple 500 can be configured to have a natural, undeformed state, an example of such state is shown at FIGS. 4A and 4B, and to transition to a deformed insertion state upon application of a load force at the staple 500. For example, the staple 500 can have the biased compression-inducing state where the first leg 502 and the second leg 504 are angled toward one another, which can help to apply a compression force to urge bones together when the staple 500 is positioned at and across such bones. Upon application of a load force to the staple 500, the staple 500 can be configured to transition from an undeformed state in which the legs of the staple are at their natural or resting positions to a deformed insertion state at which the first and second legs 502, 504 (e.g., end portion 537 of first leg 502 and end portion 538 of second leg 504) are spaced further apart (e.g., and oriented generally parallel to one another) as compared to the natural state. In particular, the staple 500 can be configured such that upon application of the load force at the staple 500, the first leg 502 is configured to move in a direction 540 (e.g., away from the bridge 506) and the second leg 504 is configured to move in a direction 542 (e.g., away from the bridge 506) from the undeformed state to the deformed insertion state. Conversely, upon reduction or removal of the applied load force at the staple 500, the staple 500 can be configured such that the first leg 502 is configured to move in a direction opposite the direction 540 (e.g., toward the bridge 506) from the deformed insertion state back toward the undeformed state and the second leg 504 is configured to move in a direction opposite the direction 542 (e.g., toward the bridge 506) from the deformed insertion state back toward the deformed state.

In use, the staple 500 can provide compression across the end faces of the bones into which the staple is inserted. Compression can occur when the legs of the staple are inserted into the bones (e.g., into pre-drilled implant openings in the bones) at a spacing and/or angle greater than the natural, undeformed configuration of the legs. The staple legs can be deformed to be inserted into the bones and, when the force applied to deform the legs is released, the staple legs can elastically bias toward their unbiased (natural or undeformed) shape. However, the spacing and/or angulation of the legs inserted into the bones can prevent the legs from fully returning to their undeformed state. As a result, the staple can apply a compressive force between the end faces of the bones into which the staple legs are inserted (e.g., with the force directed in the direction of convergence of the staple legs). The compressive force may help promote bone healing and fusion between the bones into which the staple is inserted.

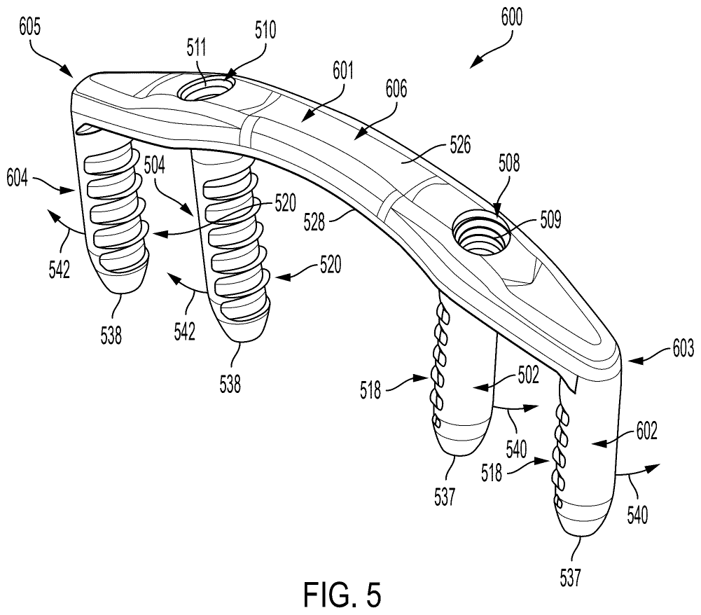

FIG. 5 is a perspective view of another embodiment of a staple 600. The staple 600 can be similar to, or the same as, the staple 500 described previously except as noted here. For example, the staple 600 can include one or more (e.g., each) of the features disclosed herein with respect to the staple 500 except as otherwise noted here.

For example, the staple 600 can include a body 601 and, in addition to the first leg 502 and the second leg 504 at the body 601, a third leg 602 and a fourth leg 604 at the body 601. For the illustrated embodiment, the staple 600 includes the first leg 502 and the third leg 602 at a first side 603 of a bridge 606, and thus of the staple 600, and the second leg 504 and the fourth leg 604 at a second side 605 of the bridge 606, and thus of the staple 600. Here the first side 603 is opposite the second side 605. The arrangement of the legs 502, 504, 602, 604 of the staple 600 can thus be a four-leg, in-line arrangement. In some examples, each of the legs 502, 504, 602, 604 can each have an equal length 512, and in the biased compression-inducing state of the staple 600 shown at FIG. 5, the bridge 606 can arch upward away from end portions 537, 538 of legs 502, 504, 602, 604 such that the end portions 537, 538 of legs 602, 604 are at a different elevation than the end portions 537, 538 of legs 502, 504. This arrangement can result in starting insertion of the legs 602, 604 into one or more bones before starting insertion of the legs 502, 504 into one or more bones (e.g., because the legs 602, 604 can contact the one or more bones first and the legs 502, 504 contact the one or more bones later after the legs 602, 604 of the staple 600 has begun inserting into the one or more bones).

The bridge 606 can connect the first and third legs 502, 602 to the second and fourth legs 504, 604. The bridge length 516 of the bridge 606 can range from 28 mm to 34 mm, which can be sufficient to allow for positioning the bridge across a space (e.g. joint) between bones in the foot while maintaining the legs 502, 602 at one bone and the legs 504, 604 at another bone with the bridge 606 placed across the spaced between such bones. As an example, the bridge length 516 for the bridge 606 can be measured from a central longitudinal axis of the outermost leg 602 at the first side 603 to a central longitudinal axis of the outermost leg 604 at the second side 605.

The staple 600 can have the first handling coupling 508, second handling coupling 510, first handling coupling receptacle 509, and second handling coupling receptacle 511 and one or more (e.g., all) of the features associated therewith as disclosed with respect to the staple 500. Furthermore, the staple 600 can have the material and teeth 518, 520 as well as be configured to transition between the biased compression-inducing state and the deformed insertion state as disclosed with respect to the staple 500.

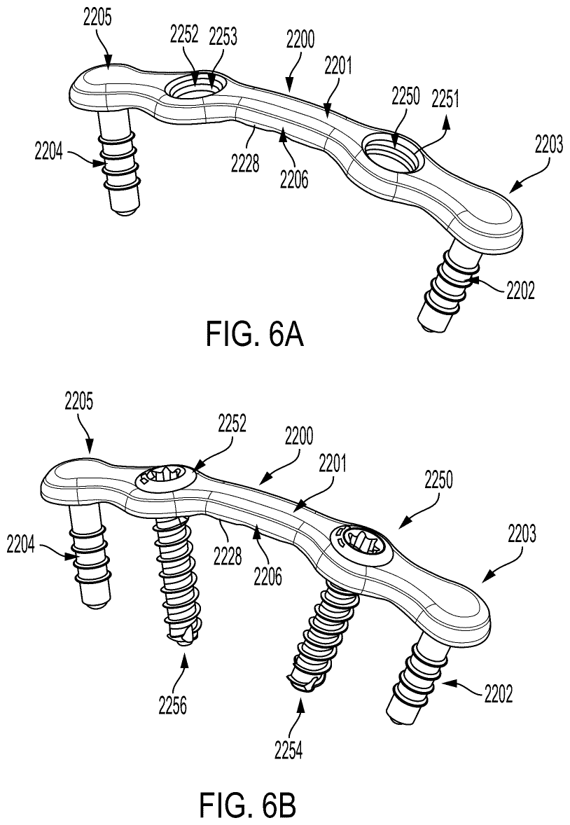

FIGS. 6A and 6B illustrate an embodiment of an orthopedic implant in the form of a staple 2200 that includes one or more fixation apertures. FIG. 6A is a perspective view of this embodiment of the staple 2200 showing fixation apertures 2250, 2252, and FIG. 6B is a perspective view of this embodiment of the staple 2200 showing fixation members 2254, 2256 placed at the fixation apertures 2250, 2252, respectively. As described elsewhere herein, the staple 2200 can be configured to apply a compression force at the bones and across the space (e.g., joint) between the bones for use in fixating and fusing bones. The staple 2200 can include any one or more features (e.g., each of the features) illustrated and/or described elsewhere herein with respect to orthopedic implants, including with respect to other staple embodiments as illustrated and/or described elsewhere herein.

The staple 2200 can include a staple body 2201 having a first leg 2202, a second leg 2204, and a bridge 2206. For the illustrated embodiment, the staple 2200 includes the first leg 2202 at a first side 2203 of the staple 2200 and the second leg 2204 at a second side 2205 of the staple 2200. In this example, the first side 2203 is opposite the second side 2205. The bridge 2206 can connect the first side 2203 and the second side 2205, and, thus, for example, connect the first leg 2202 and the first fixation aperture 2250 with the second leg 2204 and the second fixation aperture 2252.

As noted, the staple 2200 can further include one or more fixation apertures 2250, 2252. Each of the one or more fixation apertures 2250, 2252 can be configured to receive thereat (e.g., therethrough) a respective fixation member 2254, 2256 (e.g., a bone screw). The fixation aperture 2250, 2252 can receive the respective fixation member 2254, 2256 such that the respective fixation member 2254, 2256 extends through the fixation aperture 2250, 2252 and out from a bottom surface 2228 of the staple body 2201. To accommodate a respective fixation member 2254, 2256, each fixation aperture 2250, 2252 can include a respective complementary coupling element 2251, 2253 that can be configured to couple to a fixation member. For example, as illustrated here, the fixation aperture 2250 can include first complementary coupling element 2251 in the form of one of male and female threading that is complementary to and configured to couple to the other of male and female threading included at the coupling element 2254, and the fixation aperture 2252 can include second complementary coupling element 2253 in the form of male or female threading that is complementary to and configured to couple to the other of male and female threading included at the coupling element 2256. Accordingly, the fixation aperture 2250 can be configured to receive thereat and couple to fixation member 2254 (e.g., a first bone screw) and fixation aperture 2252 can be configured to receive thereat and couple to fixation member 2256 (e.g., a second bone screw). Depending on the orientation of the fixation apertures 2250, 2252 relative to the body 2201, the staple 2200 can be configured such that the fixation member 2254 extends parallel to the leg 2202 and the fixation member extends parallel to the leg 2204 or such that the fixation member 2254 extends at a skewed orientation relative to the leg 2202 and the fixation member 2256 extends at a skewed orientation relative to the leg 2204.

The staple 2200 as illustrated at FIGS. 6A and 6B includes the fixation apertures 2250, 2252 inside of (e.g., relative to a longitudinal length of the staple 2200) the legs 2202, 2204. In particular, the staple 2200 can have the leg 2202 and the fixation aperture 2250 at the first side 2203 with the fixation aperture 2250 closer to the bridge 2206 than the leg 2202 as measured along the longitudinal length of the staple 2200, and the staple 2200 can have the leg 2204 and the fixation aperture 2252 at the second side 2205 with the fixation aperture 2252 closer to the bridge 2206 than the leg 2204 as measured along the longitudinal length of the staple 2200. The leg 2202 and the fixation aperture 2250, and thus the first side 2203 of the staple 2200, can be configured to be placed at a first bone and the leg 2204 and the fixation aperture 2252, and thus the second side 2205 of the staple 2200, can be configured to be placed at a second bone (e.g., the second bone different from the first bone or a fragment/piece of the first bone), for instance as disclosed elsewhere herein to apply compression across a space separating the first and second bones.

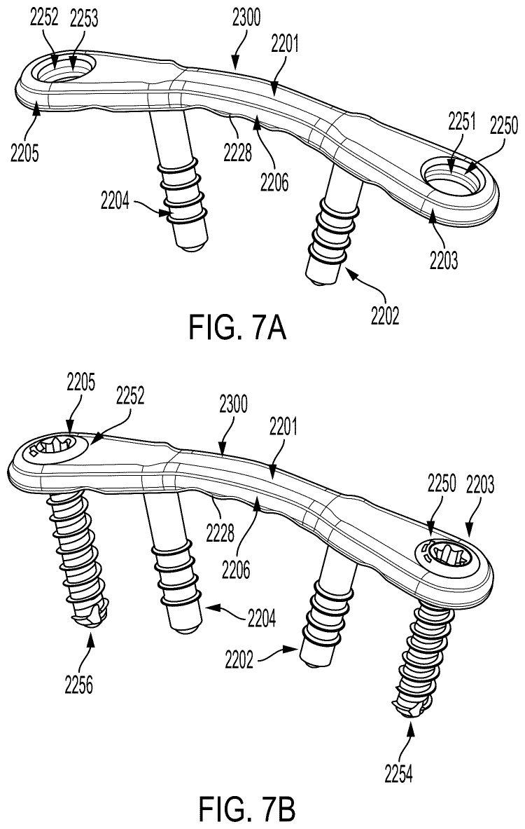

FIGS. 7A and 7B illustrate an additional embodiment of an orthopedic implant in the form of a staple 2300 that includes one or more fixation apertures. The staple 2300 can be similar to, or the same as, the staple 2200 illustrated and described with respect to FIGS. 6A, 6B except as otherwise illustrated and described here with respect to FIGS. 7A, 7B. As such, like reference characters are used to denote like elements. FIG. 7A is a perspective view of this embodiment of the staple 2300 showing the fixation apertures 2250, 2252, and FIG. 7B is a perspective view of this embodiment of the staple 2300 showing fixation members 2254, 2256 placed at the fixation apertures 2250, 2252. The staple 2300 can include any one or more features (e.g., each of the features) illustrated and/or described elsewhere herein with respect to orthopedic implants, including with respect to other staple embodiments as illustrated and/or described elsewhere herein.

Like the staple 2200, the staple 2300 includes the first leg 2202 and the fixation aperture 2250 at the first side 2203 and includes the second leg 2204 and the fixation aperture 2252 at the second side 2205. However, the staple 2300 differs from the staple 2200 in the arrangement of the legs 2202, 2204 and the fixation apertures 2250, 2252. Namely, the staple 2300 as illustrated at FIGS. 7A and 7B includes the fixation apertures 2250, 2252 outside of (e.g., relative to a longitudinal length of the staple 2300) the legs 2202, 2204. In particular, the staple 2300 can have the leg 2202 and the fixation aperture 2250 at the first side 2203 with the leg 2202 closer to the bridge 2206 than the fixation aperture 2250 as measured along the longitudinal length of the staple 2300, and the staple 2300 can have the leg 2204 and the fixation aperture 2252 at the second side 2205 with the leg 2204 closer to the bridge 2206 than the fixation aperture 2252 as measured along the longitudinal length of the staple 2300. The leg 2202 and the fixation aperture 2250, and thus the first side 2203 of the staple 2300, can be configured to be placed at a first bone and the leg 2204 and the fixation aperture 2252, and thus the second side 2205 of the staple 2300, can be configured to be placed at a second bone (e.g., the second bone different from the first bone or a fragment/piece of the first bone), for instance as disclosed elsewhere herein to apply compression across a space separating the first and second bones.

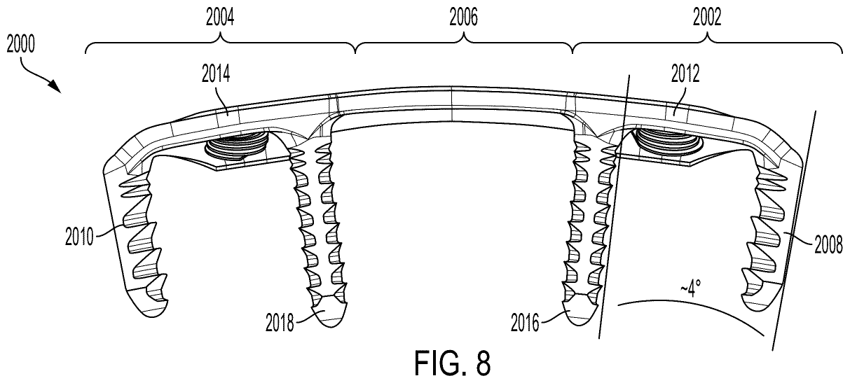

FIG. 8 is a side view of an additional embodiment of an orthopedic implant in the form of a staple 2000 that includes one or more fixation aperture. Staple 2000 can include any of the features and configurations of a staple discussed herein, including features and configurations discussed with respect to staple 2200 and staple 2300 above. In the example of FIG. 8, staple 2000 includes a body defining first side 2002, a second side 2004, and a bridge 2006 connecting the first side to the second side. First side 2002 of staple 2000 includes at least one leg 2008 (which can be referred to as first leg 2008), and second side 2004 of staple 2000 can also include at least one leg 2010 (which can be referred to as second leg 2010).

Staple 2000 can further include one or more fixation apertures, such as one or more first fixation apertures 2012 on first side 2002 of the staple and one or more second fixation apertures 2014 on second side 2004 of the staple. Each of the one or more fixation apertures can be configured to receive thereat (e.g., therethrough) a respective fixation member (e.g., a bone screw). The fixation apertures can receive the respective fixation members such that the respective fixation members extend through the fixation apertures and out from a bottom surface of the staple body. In different examples, a perimeter of one or more of the fixation apertures (e.g., each of the fixation apertures) is threaded, e.g., to threadingly engage a threaded head of a locking screw and/or to threadingly engage a drill/insertion guide.

As illustrated in FIG. 8, staple 2000 has first leg 2008 and a third leg 2016 on first side 2002 of the staple with first fixation aperture 2012 positioned between the first and third legs. In addition, staple 2000 has second leg 2010 and fourth leg 2018 on second side 2004 of the staple with second fixation aperture 2014 positioned between the second and fourth legs. The inner third and fourth legs 2016, 2018 are illustrated as having teeth on both inner and outer sides of the leg whereas the outer first and second legs 2008, 2010 are illustrated as only including teeth on the inner side of the leg.

In some examples, first leg 2008 and third leg 2016 each define a length, and the lengthwise extent of the first leg extends parallel to the lengthwise extent of the third leg. Additionally or alternatively, second leg 2010 and fourth leg 2018 can each define a length, with the lengthwise extent of the second leg extending parallel to the lengthwise extent of the fourth leg. In other examples, the lengthwise extent of first leg 2008 can extend at a non-parallel angle relative to the lengthwise extent of third leg 2016 and/or the lengthwise extent of second leg 2010 can extend at a non-parallel angle relative to the lengthwise extent of fourth leg 2018. For example, the lengthwise extent of first leg 2008 can extend at a non-parallel angle 2020 relative to the lengthwise extent of third leg 2016 and/or the lengthwise extent of second leg 2010 can extend at the same or different non-parallel angle 2020 relative to the lengthwise extent of fourth leg 2018. Non-parallel angle 2020 may angle first leg 2008 inwardly toward third leg 2016 and/or second leg 2010 inwardly toward fourth leg 2018. In some examples, angle 2020 is within a range from 1 degree to 20 degrees, such as from 2 degrees to 10 degrees, or 3 degrees to 5 degrees.

A staple according to the present disclosure can be fabricated from a variety of different materials. The staple may be fabricated from a biocompatible metal (e.g., titanium, stainless steel, nickel titanium alloy (nitinol)). In one example, the staple is fabricated from titanium (e.g., the staple is formed of a metal consisting of or consisting essential of titanium). The metal forming the staple may be substantially or entirely devoid of nickel. Titanium can be useful in that the metal can resist high energy forces without breakage and can avoid nickel sensitivity issues that may be exhibited by some patients. When so configured, the entire body of the staple (e.g., bridge, legs) can be formed of titanium. During insertion, the legs of the titanium staple may be elastically deformed, allowing the legs to return to toward their original, undeformed position. Other materials, including combinations of different materials, may be used in other configurations of a staple according to the disclosure.

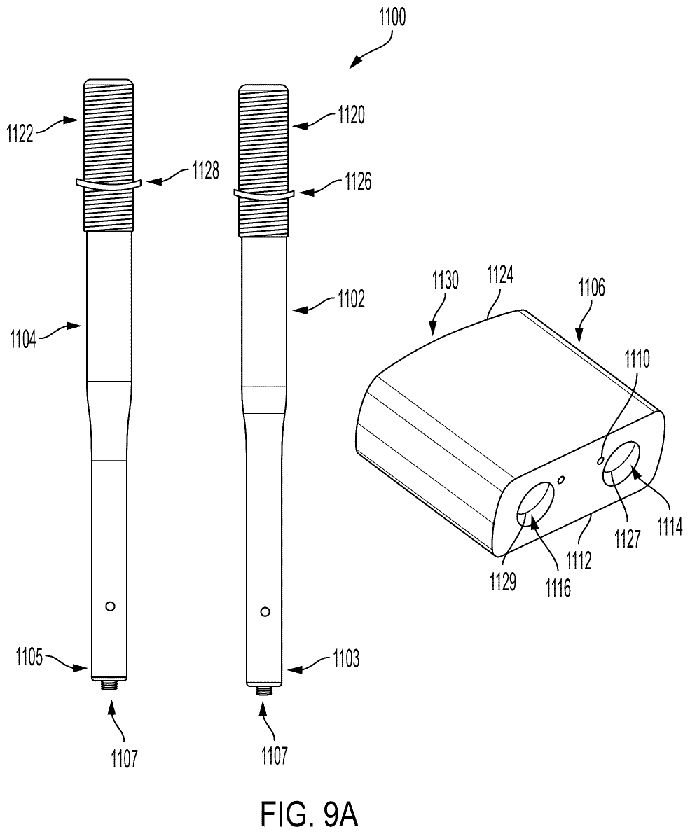

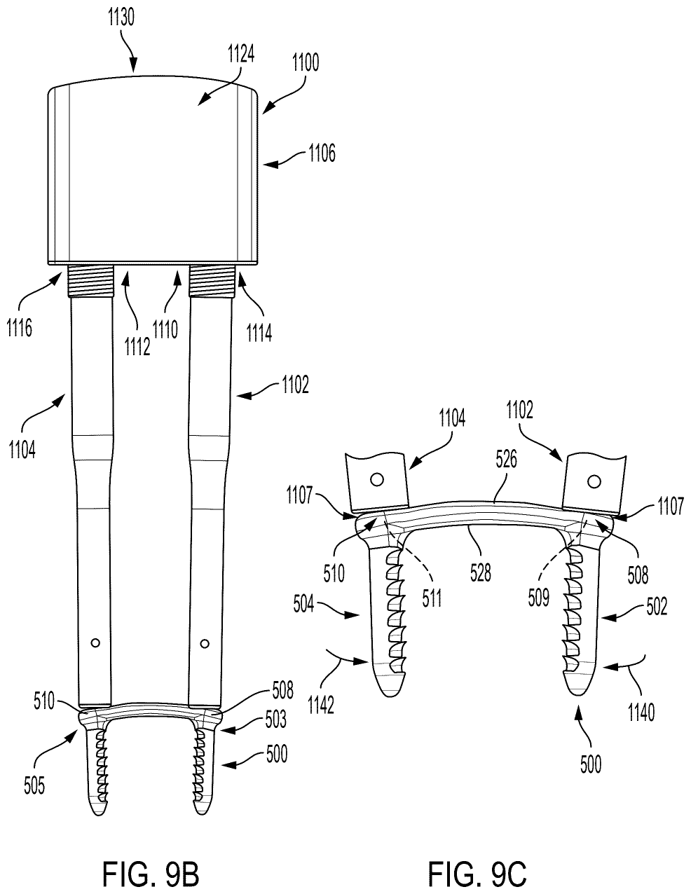

FIGS. 9A-9C illustrate an embodiment of an inserter 1100. FIG. 9A shows disassembled components of the inserter 1100, FIG. 9B shows assembled components of the inserter 1100, and FIG. 9C shows the inserter 1100 operatively connected to staple 500 with a load force removed such that the staple 500 is in its biased, compression state. Inserter 1100 is described in conjunction with FIGS. 9A-9C as being operatively connected to staple 500, although inserter 1100 can be operatively connected to a staple having any configuration as described herein. Accordingly, discussion of the relationship between inserter 1100 and staple 500 is equally applicable to staples having other configurations, and features described in relation to staple 500 can be substituted with corresponding features of other staple configurations according to the disclosure.

The inserter 1100 can include a first coupling shaft 1102, a second coupling shaft 1104, and a connector 1106. The first and second coupling shafts 1102, 1104 can be configured to operatively connect to an implant, such as a staple. The illustrated embodiment of the inserter 1100 shows the first and second coupling shafts 1102, 1104 each configured to operatively couple to the staple 500 (or another staple configuration, such as any of those as described elsewhere herein). The connector 1106 can be configured to join the first coupling shaft 1102 and the second coupling shaft 1104, for instance as shown at the example of FIG. 9B

In particular, the first coupling shaft 1102 can be configured to operatively couple to the staple 500 at the first handling coupling 508, and the second coupling shaft 1104 can be configured to operatively couple to the second handling coupling 510. For example, the first coupling shaft 1102 can have a distal end portion 1103 and the second coupling shaft 1104 can have a distal end portion 1105, and each of such distal end portions 1103, 1105 can include an implant coupling member 1107. The coupling member 1107 at the distal end portion 1103 of the first coupling shaft 1102 can be configured to operatively connect to a complementary coupling member at the first handling coupling 508, and the coupling member 1107 at the distal end portion 1105 of the second coupling shaft 1104 can be configured to operatively connect to a complementary coupling member at the second handling coupling 510. In this way, the inserter 1100 can include the first coupling shaft 1102 connected to a first side of the implant, such as the first side 503 of the staple 500, and the second coupling shaft 1104 connected to a second side of the implant, such as the second side 505 of the staple 500.

As one such specific example, each of the first and second handling couplings 508, 510 can include threading as a type of complementary coupling member thereat. The coupling member 1107 at the distal end portions 1103, 1105 of the respective first and second coupling shafts 1102, 1104 can include threading that is configured to operatively couple to the complementary threading at the respective first and second handling couplings 508, 510. Thus, in this particular example, operatively coupling the inserter 1100 to an implant, such as the staple 500, can include threadingly inserting the first coupling shaft 1102 into the first handling coupling 508 (e.g., from the top surface 526 of the staple 500) and threadingly inserting the second coupling shaft 1104 into the second handling coupling 510 (e.g., from the top surface 526 of the staple 500). Other types of mechanical connections than threading can be used.

As best seen at FIG. 9C, each of the first coupling shaft 1102 and the second coupling shaft 1104 can be configured to couple to the respective first and second handling coupling receptacles 509, 511 at a location between the top surface 526 of the staple 500 and the bottom surface 528 of the staple 500. For example, the first coupling shaft 1102 can operatively couple to the first handling coupling 508 from the top surface 526 and in a direction toward the bottom surface 528 but without the first coupling shaft 1102 extending out from the bottom surface 528. Likewise, the second coupling shaft 1104 can operatively couple to the second handling coupling 510 from the top surface 526 and in a direction toward the bottom surface 528 but without the second coupling shaft 1104 extending out from the bottom surface 528. As one such example, the coupling member 1107 at the first coupling shaft 1102 can extend within the first handling coupling receptacle 509 such that a distal end of the coupling member 1107 at the first coupling shaft 1102 is contained within the staple 500 (e.g., within the first handling coupling receptacle 509). Similarly, the coupling member 1107 at the second coupling shaft 1104 can extend within the second handling coupling receptacle 511 such that a distal end of the coupling member 1107 at the second coupling shaft 1104 is contained within the staple 500 (e.g., within the second handling coupling receptacle 511).

In a further embodiment, to help provide added stability when applying a load force at the implant (e.g., staple 500), the first and/or second coupling shaft 1102, 1104 can include a shaft stabilizing arm. When so included, the shaft stabilizing arm can be included at the respective distal end portion 1103, 1105 of the respective coupling shaft 1102, 1104 and extend in a direction parallel to a central longitudinal axis of the respective coupling shaft 1102, 1104. Where the implant is a staple, the shaft coupling arm can be configured to contact the bridge of the staple (e.g., at a top and/or side surface of the bridge but not a bottom surface of the bridge) when the coupling member 1107 of the respective shaft 1102, 1104 is at the respective handling coupling at the staple. The inclusion of such shaft stabilizing arm can be useful to help provide additional stability during the application of a load force at the implant (e.g., staple) and placement of the implant (e.g., staple) at the target anatomy.

In some examples, such as the embodiment illustrated at FIGS. 9A-9C, the inserter 1100 can further include a first wire receiving opening 1110, a second wire receiving opening 1112, a first receptacle 1114, and a second receptacle 1116. The first wire receiving opening 1110 can be configured to receive a first wire, and the second wire receiving opening 1112 can be configured to receive a second wire. The first receptacle 1114 can be configured to receive and hold the first coupling shaft 1102, for instance a proximal end portion 1120 of the first coupling shaft 1102, and the second receptacle 1116 can be configured to receive and hold the second coupling shaft 1104, for instance a proximal end portion 1122 of the second coupling shaft 1104. For the illustrated embodiment, each of the first wire receiving opening 1110, the second wire receiving opening 1112, the first receptacle 1114, and the second receptacle 1116 is included at the connector 1106.

As noted, the connector 1106 can be configured to join the first coupling shaft 1102 and the second coupling shaft 1104, for instance as shown at the example of FIG. 9B. As one example, the connector 1106 can be configured to join the first and second coupling shafts 1102, 1104 by receiving the first coupling shaft 1102 at the first receptacle 1114 at the connector 1106 and receiving the second coupling shaft 1104 at the second receptacle 1116 at the connector 1106. As such, with the receptacles 1114, 1116 at the connector 1106 receiving and holding the respective coupling shaft 1102, 1104, the connector 1106 can removably join the coupling shafts 1102, 1104. The receptacles 1114, 1116 at the connector 1106 are illustrated here at a bottom surface of the connector 1106, and in other embodiments the receptacles 1114, 1116 can be at other locations at the connector 1106, such as receptacle 1114 extending along one side surface of the connector 1106 and the receptacle 1116 extending along another (e.g., opposite) side surface of the connector 1106.

To help facilitate a more robust joining of the coupling shafts 1102, 1104 via the connector 1106, the first coupling shaft 1102 can include a first retention feature 1126 and the second coupling shaft 1104 can include a second retention feature 1128. For example, the first retention feature 1126 can be located at or near the proximal end portion 1120 of the first coupling shaft 1102, and the second retention feature 1128 can be located at or near the proximal end portion 1122 of the second coupling shaft 1104. The first retention feature 1126 can be configured to help hold the first coupling shaft 1102 in the first receptacle 1114 and the second retention feature 1128 can be configured to help hold the second coupling shaft 1104 in the second receptacle 1116. In one such further example, the connector 1106 can include a first retention mating feature 1127 at the first receptacle 1114 and a second retention mating feature 1129 at the second receptacle 1116. The first retention mating feature 1127 can be complementary to the first retention feature 1126 and configured to receive and hold the first retention feature 1126, and the second retention mating feature 1129 can be complementary to the second retention feature 1128 and configured to receive and hold the second retention feature 1128. The first retention feature 1126 and the first retention mating feature 1127 as well as the second retention feature 1128 and the second retention mating feature 1129 can take any of a variety of suitable forms of complementary connector pairs, such as, for one suitable, non-limiting example, complementary structures that create an interference fit. As one such specific example, the first retention feature 1126 and the first retention mating feature 1127 as well as the second retention feature 1128 and the second retention mating feature 1129 can be complementary connector pairs that provide a retention force in a direction generally parallel to a longitudinal axis of the coupling shafts 1102, 1104 and configured to release this retention force upon movement of at least one of the complementary connectors of the pair (e.g., movement of one of the coupling shaft 1102 and the receptacle 1114 and movement of one of the coupling shaft 1104 and the receptacle 1116) in a direction generally transverse to longitudinal axis of the coupling shafts 1102, 1104.

As one specific such example, the connector 1106 as shown for the illustrated embodiment of the inserter 1100 can include a cap 1124. For the illustrated embodiment of the inserter 1100, the cap 1124 can be configured to be positioned over the proximal end portion 1120 (e.g., opposite the implant, such as the staple 500) of the first coupling shaft 1102 and over the proximal end portion 1122 (.g., opposite the implant, such as the staple 500) of the second coupling shaft 1104. For instance, the cap 1124 can be configured to join the first and second coupling shafts 1102, 1104 by placing the first receptacle 1114 at the cap 1124 over the proximal end portion 1120 of the first coupling shaft 1102 and the second receptacle 1116 at the cap 1124 over the proximal end portion 1122 of the second coupling shaft 1104, and then moving the cap 1124 as so positioned relative to the first and second coupling shafts 1102, 1104 (e.g., in a direction toward one or more bones) such that the proximal end portions 1120, 1122 are received and held at the respective receptacles 1114, 1116. Likewise, in certain embodiments of the cap 1124 that include wire receiving openings, the cap 1124 can similarly be configured to receive and hold first and second wires, positioned at one or more bones, at respective first and second wire receiving openings 1110, 1112 as the cap 1124 is moved relative to such wires (e.g., in a direction toward one or more bones).

The cap 1124 can further include a surface contour 1130. The surface contour 1130 can be adapted to fit at a hand of a user. For the illustrated example, the surface contour 1130 can have a highest elevation at a location between the receptacles 1114, 1116 and a lowest elevation outside of the receptacles 1114, 1116 such that the surface contour 1130 angles downward toward the implant, such as the staple 500, when moving along the surface contour 1130 away from the highest elevation between the receptacles 1114, 1116. Accordingly, when positioning the implant, such as the staple 500, in contact with the first bone and the second bone, a user's hand can tamp at the surface contour 1130 of the cap 1124 to apply insertion force at the implant, such as the staple 500.

The inserter 1100 can be configured to place the implant, such as the staple 500, in one or more bones (e.g., using an implant guide sleeve to guide advancement of the inserter 1100 and thereby guide positional placement of the staple 500 into one or more bones). For example, the inserter 1100 can be operatively connected to the staple 500 via the first and second coupling shafts 1102, 1104, and the connector 1106 (e.g., cap 1124) can be joined to the first and second coupling shafts 1102, 1104, such as shown at the example of FIG. 9B. When the connector 1106 is joined to the first coupling shaft 1102 and the second coupling shaft 1104, such as shown at the example of FIG. 9B, the connector 1106 can be configured to bias the first coupling shaft 1102 and the second coupling shaft 1104 toward each other to apply a load force to the staple 500. When the first coupling shaft 1102 and the second coupling shaft 1104 are so biased toward each other to apply the load force to the staple 500, the first leg 502 and the second leg 504 can be oriented generally parallel to one another, such as shown at the example of FIG. 9B. This application of the load force to the staple 500 can cause the legs 502, 504 to move away from each other to the generally parallel orientation which can be a configuration useful for inserting the staple 500 in and/or across the bones. Then, after the staple 500 has been inserted as desired at and across the bones, the connector 1106 can be removed from at least one of the first coupling shaft 1102 and the second coupling shaft 1104 to cause, as shown at the example of FIG. 9C, the first leg 502 and the second leg 504 to move toward one another. Specifically, removing the connector 1106 can cause the first leg 502 to move in a direction 1140 toward the second leg 504 and cause the second leg 504 to move in a direction 1142 toward the first leg 502.

During use of a staple and staple insertion system, as described herein, a clinician may first drill holes into underlying first and second bone portions aligned with the location of the one or more legs of the staple being inserted. With the staple energized (e.g., by biasing the first and second coupling shafts 1102, 1104 toward each other and provisionally fixing the position of the shafts, and correspondingly staple legs, relative to each other with connector 1106), the clinician can insert the legs of the staple into the drilled holes. Before or after, the clinician may drill holes into underlying first and second bone portions aligned with fixation apertures of the staple and guide fixation members (e.g., screw) through the fixation apertures of the staple and into the underlying bone portions.

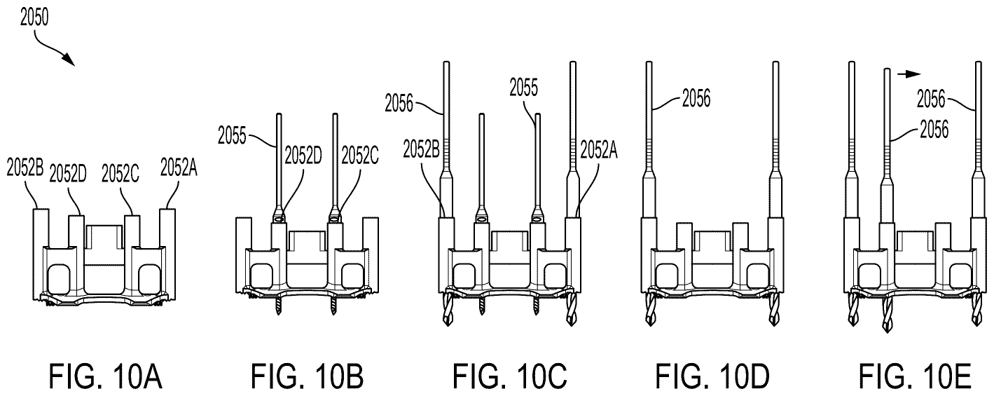

FIGS. 10A-10E illustrate an example staple leg drill guide 2050 having one or more guide openings, each opening being configured to guide drilling of a hole into an underlying bone at a location where a leg of a staple is to be inserted. In the illustrated configuration, staple leg drill guide 2050 includes four staple leg guide openings, including a first staple leg guide opening 2052A, a second staple leg guide opening 2052B, a third and staple leg guide opening 2052C, and a fourth staple leg guide opening 2052D. Each staple leg guide opening may define a partially or fully enclosed lumen or tube configured to guide a cutting member (e.g., drill) for forming and an opening in the bone underlying the opening. The number and arrangement of drill guide openings may vary to correspond to the number and arrangement of staple legs on the staple being inserted into underlying bones. Staple leg drill guide 2050 can be used for forming four staple leg openings, e.g., for inserting four staple legs such as illustrated with staple 2000 in FIG. 8.

Staple leg drill guide 2050 can include a body 2054 mimicking the shape and configuration of the staple (e.g., staple 2000) to be inserted at the location prepared using the drill guide. For example, body 2054 can include a size (e.g., length, width, and/or thickness) and shape that substantially mimics or corresponds to the configuration of the staple (e.g., staple 2000) to be inserted at the location prepared using the drill guide. This allows the clinician to visualize how and where the staple will be positioned upon installation. Each staple leg guide opening of drill guide 2050 may be defined by a lumen extending axially away from body 2054 of the drill guide and/or a lumen extending through the thickness of the drill guide.

In use, the clinician can position staple leg drill guide 2050 at a location where predrilled holes are to be formed in the underlying bone portions to facilitate installation of the legs of the staple. For example, the clinician can position staple leg drill guide 2050 with one or staple leg guide openings 2052A, 2052C positioned over a phalanx and one or more other staple leg guide openings 2052B, 2052D position over a metatarsal, with the staple leg drill guide spanning across an MTP joint.

With reference to FIG. 10B, the clinician can temporarily fixate staple leg drill guide 2050 to one or both underlying bone portions. For example, the clinician may insert pins or fixation wires 2055 through the inner two staple leg guide openings 2052C, 2052D and into the underlying bones. With reference to FIG. 10C, the clinician can then insert one or more drill bits 2056 through the outer two staple leg guide openings 2052A, 2052B to form first and second staple leg holes in the bones underlying the guide openings. The clinician can then remove the fixation wires 2055 from the inner staple leg guide openings, as shown in FIG. 10D, and insert a drill bit through the inner two staple leg guide openings 2052C, 2052D to form third and fourth staple leg holes in the bones underlying the guide openings. As shown in FIG. 10E, the clinician can leave the drill bits 2056 in the outer two staple leg guide openings and/or insert temporary fixation pins or wires through the outer staple leg guide openings to secure the drill guide to the underlying bones while preparing the inner staple leg guide openings.

While FIGS. 10A-10E illustrate one example configuration in order in which staple leg holes can be formed corresponding to the number and position of staple legs to be inserted, a different order of preparation or arrangement of staple leg holes may be formed without departing from the scope of the disclosure. In general, the clinician may temporarily secure drill staple leg drill guide 2050 to one or both bone portions (e.g., proximal phalanx 250 and first metatarsal 210) using a pin, wire, and/or drill bit inserted through a staple leg guide opening into the corresponding bone portion(s) and drill through one or more other staple leg guide openings while so temporarily secured.

After preparing a suitable number and arrangement of staple leg holes in the underlying bones, the clinician can insert the legs of the staple being installed into the prepared staple leg holes. The clinician can subsequently prepare additional holes into one or both underlying bones at a location corresponding to the one or more fixation apertures of the staple being installed and insert screws through the one or more fixation apertures and into the predrilled holes.

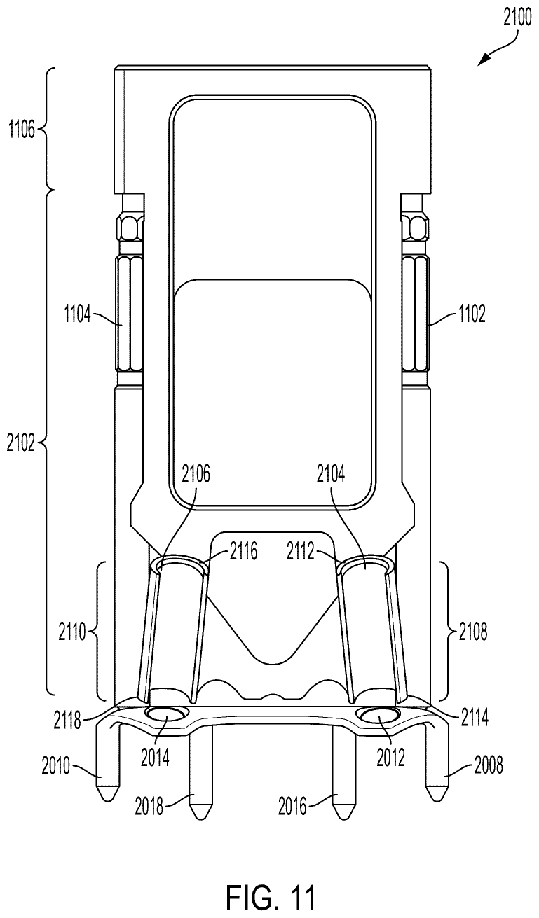

FIG. 11 illustrates an example staple implant insertion device 2100 that is configured to engage a first coupling shaft 1102 connectable to a first handling coupling through the top surface of staple 2000 and a second coupling shaft 1104 connectable to a second handling coupling through the top surface of the staple. Staple implant insertion device 2100 in FIG. 11 includes connector 1106 that joins the first coupling shaft and the second coupling shaft together, when the first coupling shaft and the second coupling shaft are biased or moved to apply a load force at staple 2000 to cause first leg 2008 and second leg 2010 to move apart from one another (in addition to causing third leg 2016 and forth leg 2018 to move apart from one another). While staple implant insertion device 2100 is illustrated and described in conjunction with staple 2000, the device can be implemented using other staple configurations as described herein.

In the example of FIG. 11, staple implant insertion device 2100 also includes a guide body 2102 operatively connected to connector 1106. In different examples, guide body 2102 and connector 1106 may be integrally formed together to provide a unitary (e.g., monolithic) structure or may be formed as separate components joined together via a mechanical coupling or linkage. In either case, guide body 2102 can define at least one guide opening, which in the illustrated example, is shown as a first guide opening 2104 and a second guide opening 2106. Each guide opening 2104, 2106 can be configured to guide drilling a hole into an underlying bone aligned with a fixation aperture extending through staple 2000 and/or to guide insertion of a screw into the underlying bone through the fixation aperture. The number and arrangement of guide openings can depend on the number and arrangement of fixation apertures defined by staple 2000.

In the illustrated example, first guide opening 2104 can be configured to guide drilling of a first hole into the first bone aligned with first fixation aperture 2012 of staple 2000 and/or insertion of a first screw into the first bone through the first fixation aperture. Second guide opening 2106 can be configured to guide drilling a second hole into a second bone aligned with second fixation aperture 2014 of staple 2000 and/or insertion of a second into the second bone through the second fixation aperture. Each guide opening 2104, 2106 may be aligned with a corresponding fixation aperture of staple 2000 such that a center axis of the opening is substantially co-linear with a center axis of the fixation aperture.

Guide body 2102 can include a first guide lumen 2108 defining first guide opening 2104 and a second guide lumen 2110 defining second guide opening 2106. First guide lumen 2108 can extend from a first end 2112 to a second end 2114, where the second end is positioned closer to staple 2000 than the first end. Second guide lumen 2110 can also extend from a first end 2116 to a second end 2118, where the first end is positioned closer to staple 2000 than the second end. Each guide lumen, and the guide opening defined by the lumen, can be sized to receive a hole forming instrument (e.g., drill bit, pin, and/or other cutting instrument) for forming an opening in the bone underlying fixation aperture 2012, 2014 and/or for guiding a screw into the bone through the fixation aperture (e.g., such that the shaft of the screw is lodged in the bone and the head of the screw contacts staple 2000 about the perimeter of the fixation aperture). It should be appreciated that example devices describe as being suitable for guiding a drill bit in conjunction with a drilling step may, in other examples, be performed using other hole forming instruments that do or do not involve rotary drilling.

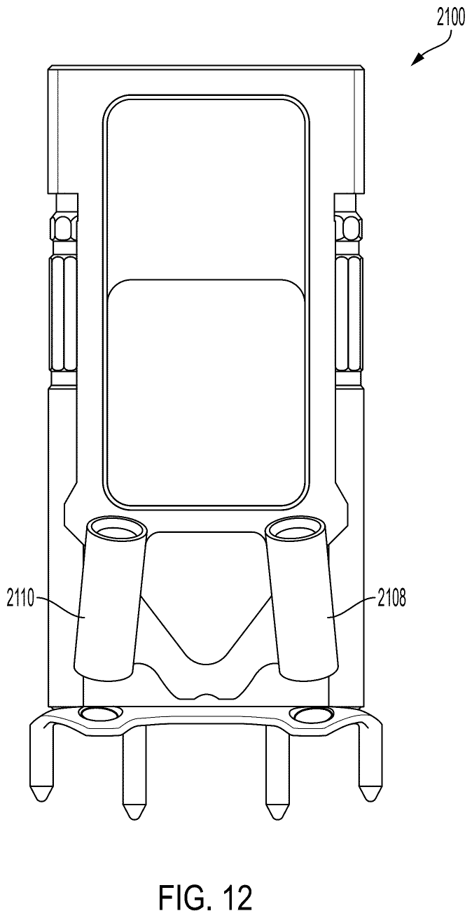

In some examples, first guide lumen 2108 and second guide lumen 2110 define a closed sidewall extending about a perimeter of the first guide lumen and second guide lumen, respectively, over at least a portion of the length of the first guide lumen and second guide lumen. For example, FIG. 12 is a side view of staple implant insertion device 2100 showing an example configuration of the device where the guide lumens are defined by closed sidewalls. As illustrated, the guide lumens are shown as circular tubes defining circular guide openings although can define other shapes in different configurations. In other configurations, such as illustrated in FIG. 11, first guide lumen 2108 and second guide lumen 2110 can have an open sidewall that extends partially but not fully about a perimeter of the guide lumen. In some examples, the guide lumen may include a slit or opening extending partially or fully along the length of the lumen. Such an opening may be useful to facilitate insertion and/or removal of a supplemental guide tube from the lumen during use. A longitudinal slit or opening extending partially but not fully along a length of a closed sidewall may also be useful for visualization inside of the lumen during use of the device.

In some examples, the clinician uses first guide lumen 2108 and second guide lumen 2110 to guide a drill bit to drill first and second holes aligned with fixation apertures 2012 and 2014, respectively. The clinician can then insert first and second screws by guiding the screw through the first and second guide lumens, respectively, through fixation apertures 2012, 2014 and into the predrilled holes in the underlying bones. To increase the precision and accuracy with which holes are drilled in the underlying bones, a clinician may use removable guide tubes having a smaller cross-sectional area (e.g., diameter) than that defined by the guide lumens.

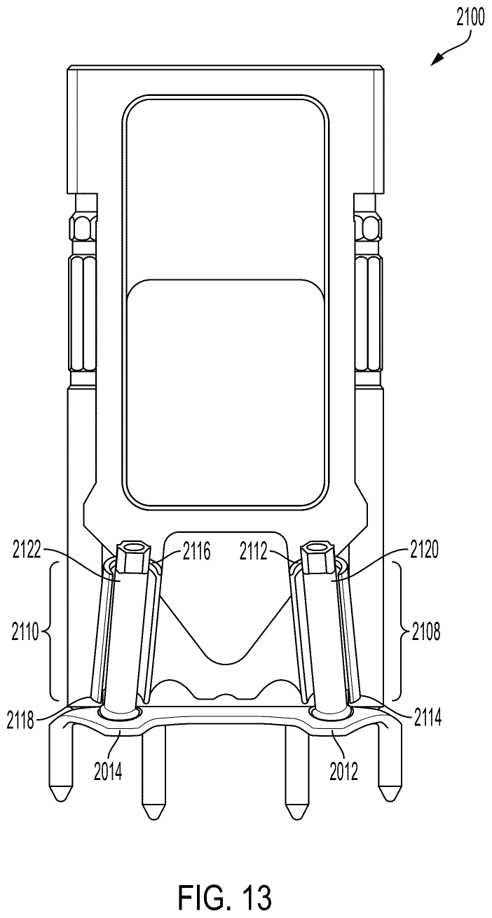

FIG. 13 is a side view of staple implant insertion device 2100 showing an example configuration of guide body 2102 utilizing guide tubes with the guide body. In particular, FIG. 13 illustrates a first guide tube 2120 at least partially inserted into first guide lumen 2108 and a second guide tube 2122 at least partially inserted into second guide lumen 2110. In some examples, each guide tube extends beyond a second end 2114, 2118 of a respective guide lumen into which the guide tube is inserted. For example, each guide tube may extend down to and/or through a top surface of staple 2000. For example, each guide tube may be inserted partially or fully into a corresponding fixation aperture of staple 2000 to more precisely align the guide tube (e.g., an axial center of an opening extending through the guide tube) with the fixation aperture (e.g., an axial center of the fixation aperture).

In some examples, first fixation aperture 2012 includes a threaded perimeter and first guide tube 2120 is configured to be threadingly engaged with the first fixation aperture of the staple. Similarly, second fixation aperture 2014 can include a threaded perimeter and second guide tube 2122 can be configured to be threadingly engaged with the second fixation aperture. In other examples, first guide tube 2120 and second guide tube 2122 may include an end region insertable into a corresponding fixation aperture of staple 2000 without mechanically engaging the fixation aperture.

In use, first guide tube 2120 can be at least partially inserted into first guide lumen 2108 and optionally engaged with first fixation aperture 2012. The first guide tube 2120 can then be used to guide a drill bit to form a first hole into the first bone aligned with first fixation aperture 2012. Thereafter, first guide tube 2120 can be removed from first guide lumen 2108 (e.g., through the first end 2112 of the lumen and/or slot opening extending through the sidewall of the lumen). With first guide tube 2120 removed, the clinician can then guide a first screw through first guide lumen 2108 and into first fixation aperture 2012 in staple 2000 and the hole formed in the underlying bone via pre-drilling.

Similarly, second guide tube 2122 can be at least partially inserted into second guide lumen 2110 and optionally engaged with second fixation aperture 2014. The second guide tube 2122 can then be used to guide a drill bit to form a second hole into the second bone aligned with second fixation aperture 2014. Thereafter, second guide tube 2122 can be removed from second guide lumen 2110 (e.g., through the first end 2116 of the lumen and/or slot opening extending through the sidewall of the lumen). With second guide tube 2122 removed, the clinician can then guide a second screw through second guide lumen 2110 and into second fixation aperture 2014 in staple 2000 and the hole formed in the underlying bone via pre-drilling.

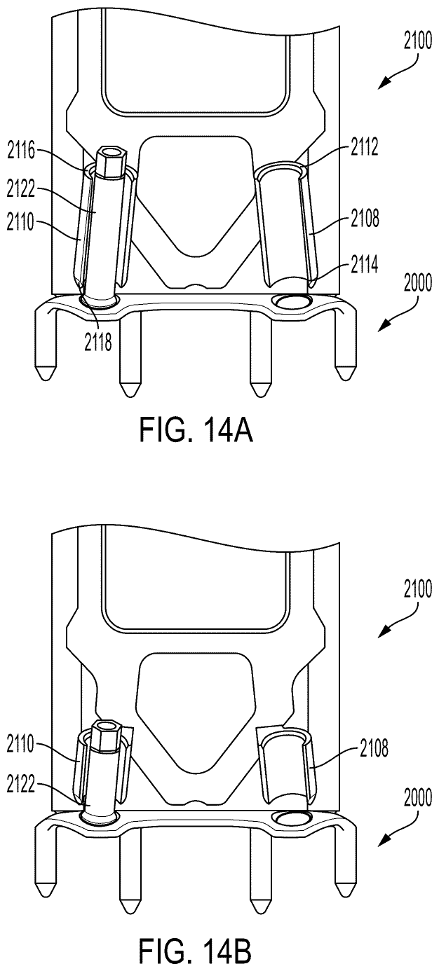

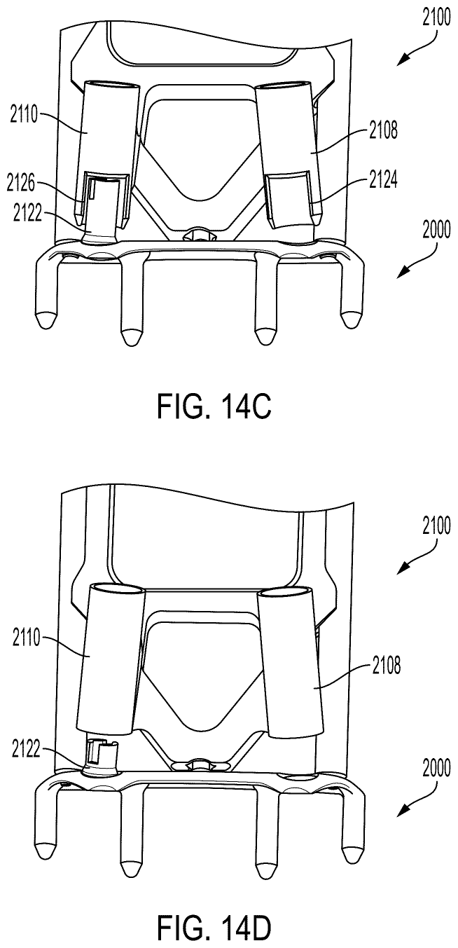

FIGS. 14A-14D illustrate a variety of different example guide tube and guide lumen configurations that can be used on staple implant insertion device 2100. FIG. 14A illustrates a configuration in which first guide tube 2120 projects beyond the first end 2112 and the second end 2114 of first guide lumen 2108 and second guide tube 2122 projected beyond the first end 2116 and the second end 2118 of the second guide lumen 2110. The first guide tube 2120 and second guide tube 2122 are configured to threadingly engage in fixation apertures 2012, 2014. After drilling holes in the bones by advancing a drill bit through the guide tubes, the guide tubes can be removes and first and second guide lumens 2108, 2010 used to guide insertion of fixation screws through the fixation apertures and into the underlying bones. FIG. 14B illustrates a similar configuration as FIG. 14A but one in first guide lumen 2108 and second guide lumen 2010 have a shorter overall length and, correspondingly, first guide tube 2120 and second guide tube 2122 can have a shorter overall length.

FIG. 14C illustrates another example configuration in which first guide lumen 2108 and second guide lumen 2110 include an upper region defining a closed sidewall and a lower region defining an open sidewall with a slot 2124, 2126 extending lengthwise along a portion of the sidewall. First guide tube 2120 is positionable within the slotted length of first guide lumen 2108, and second guide tube 2122 is also positionable within the slotted length of the second guide lumen 2110. FIG. 14D illustrates another example configuration in which first guide tube 2120 and second guide tube 2122 are positionable co-axially aligned with first guide lumen 2108 and second guide lumen 2110, respectively, but offset from the second end 2114, 2118 of the guide tube.

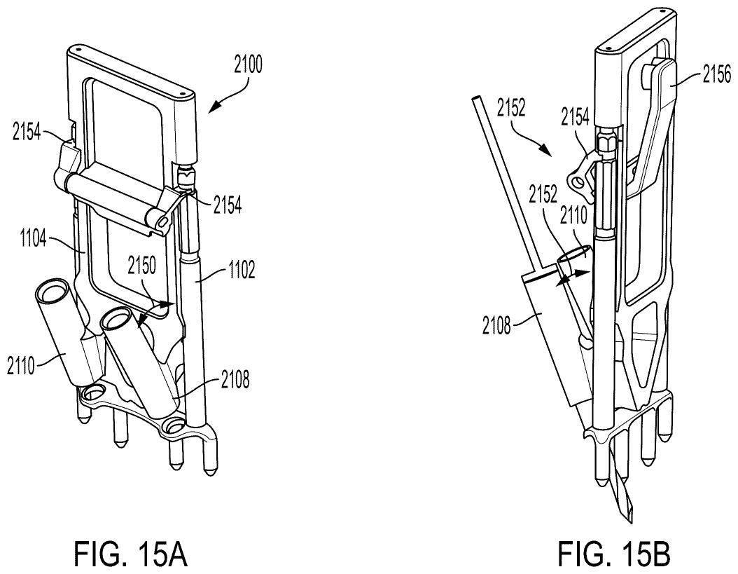

FIGS. 15A and 15B are different perspective views of example configurations of staple implant insertion device 2100. As shown, guide body 2102 can extend parallel to first coupling shaft 1102 and second coupling shaft 1104. For example, guide body 2102 may define one or more structural members extending between the two coupling shafts. The guide body 2102 can define a substantially planar face in a plane parallel to first coupling shaft 1102 and second coupling shaft 1104. First guide lumen 2108 and second guide lumen 2110 can extend angularly outwardly from the substantially planar face at an angle 2150. Angle 2150 may be within a range from 15 degrees to 85 degrees, such as from 30 degrees to 60 degrees.

In some configurations, such as that illustrated in FIGS. 15A and 15B, connector 1106 and/or guide body 2102 can be releasably locked to first coupling shaft 1102 and/or second coupling shaft 1104 to form a more stable mechanical connection between the components and the coupling shafts. For example, staple implant insertion device 2100 may include a lock 2152 that is configured to lock connector 1106 and/or guide body 2102 to first coupling shaft 1102 and/or second coupling shaft 1104. Lock 2152 may engage a detent or other surface feature on first coupling shaft 1102 and/or second coupling shaft 1104. Lock 2152 may be actuatable to releasably engage and disengage a locking connection. For example, lock 2152 may include prongs 2154 that engage detents on the coupling shafts, which are illustrated as recessed regions of the coupling shafts. The prongs 2154 may be connected via a rotating connection to an actuator 2156 that can be actuated (e.g., pushed) to engage and/or disengage the prongs from the coupling shafts.

In use, a clinician can prepare first, second, third, and fourth staple leg openings by drilling holes through corresponding first, second, third, and fourth staple leg guide openings of staple leg drill guide 2050. The clinician can then insert the first, second, third, and fourth legs of staple 2000 into the holes with coupling shafts attached to the staple energized and joined by a connector. The clinician can then use a guide body operatively connected to the connector to drill first and second holes aligned with first and second fixation apertures of staple 2000. In some examples, the clinician guides a drill bit through guide tubes inserted into guide body openings of the guide body and then removes the guide tubes from the guide body openings. In either case, the clinician can then guide screws through the guide openings defined by the guide body and into the fixation apertures of staple 2000.

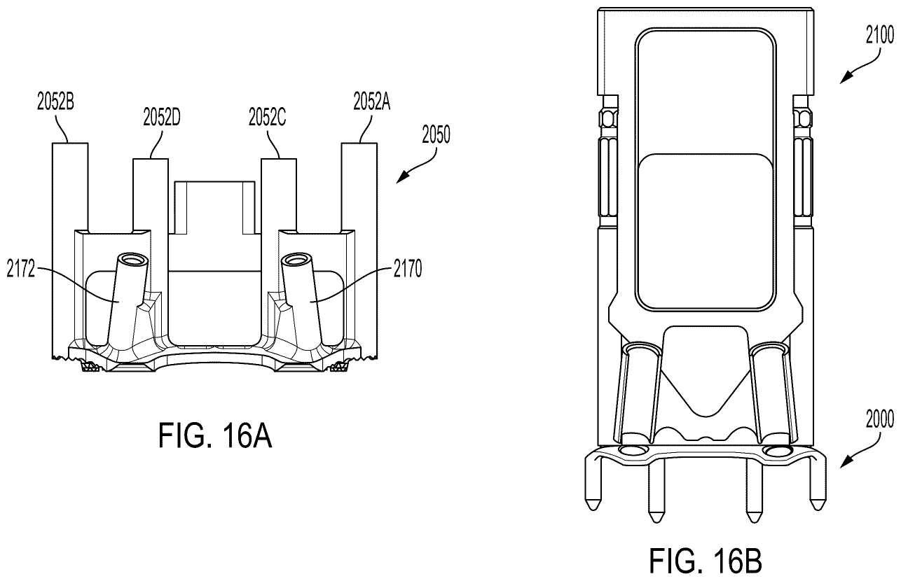

FIG. 16A is an alternative configuration of an example drill guide 2050 that is similar to the drill guide 2050 described with respect to FIGS. 10A-10E. Drill guide 2050 in FIG. 16A includes staple leg guide openings (e.g., a first staple leg guide opening 2052A, a second staple leg guide opening 2052B, a third and staple leg guide opening 2052C, and a fourth staple leg guide opening 2052D) to guide a drill bit to form openings at locations corresponding to the staple legs of staple 2000. Drill guide 2050 in FIG. 16A also includes a first and second screw guide openings 2170 and 2172 positioned at a location corresponding t the first and second fixation apertures 2012, 2014 of staple 2000. A clinician can use first and second screw guide openings 2170 and 2172 to guide a drill bit to form openings in the underlying bone at locations corresponding to the subsequent positioning of the fixation apertures of staple 2000. In some examples, the clinician inserts temporary fixation pins or wires through first and second screw guide openings 2170 and 2172 to secure drill guide 2050 to the underlying bones before or after drilling the screw leg openings and then removes the temporary fixation before guiding a drill bit to form openings in the underlying bone at locations corresponding to the subsequent positioning of the fixation apertures of staple 2000. The clinician can insert temporary fixation pins or wires through one or more staple leg guide openings of drill guide 2050 to secure the guide while guiding a drill bit through first and second screw guide openings 2170 and 2172.

FIG. 16B illustrates an example configuration of staple implant insertion device 2100 that can be used after forming openings in the bones using drill guide 2050 in the example of FIG. 16A. The clinician can insert the first, second, third, and fourth legs of staple 2000 into the pre-drilled holes with coupling shafts attached to the staple energized and joined by a connector. The clinician can then use a guide body operatively connected to the connector to guide screws through the guide openings defined by the guide body and into the pre-drilled holes aligned with the fixation apertures of staple 2000.

A staple implant insertion device according to the disclosure can have a variety of different features and configurations. For example, the number and arrangement of guide openings defined by the staple implant insertion device can vary and be positioned depending on the number and positioning of fixation apertures of the staple to be installed using the staple implant insertion device. In various examples, the staple may have one, two, three, four or more fixation apertures, and the staple implant insertion device used to install the staple can have one, two, three, four or more corresponding guide openings.

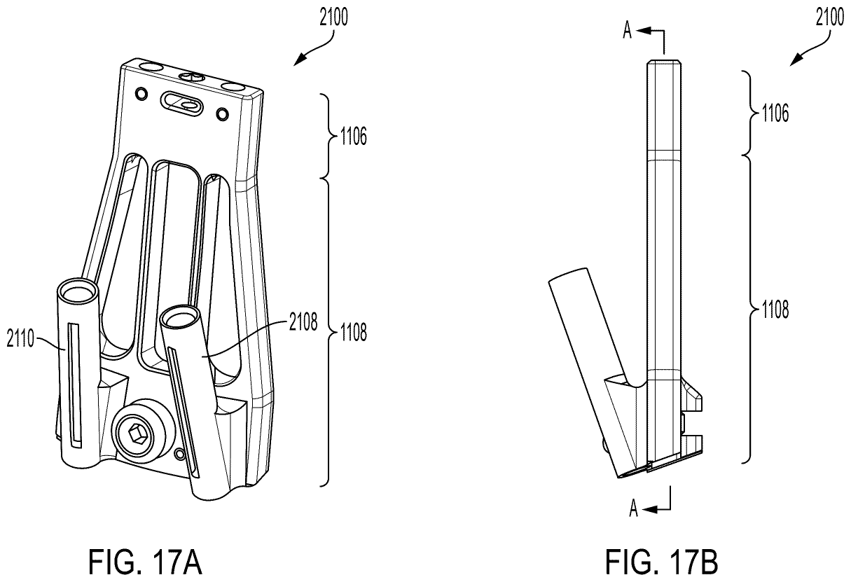

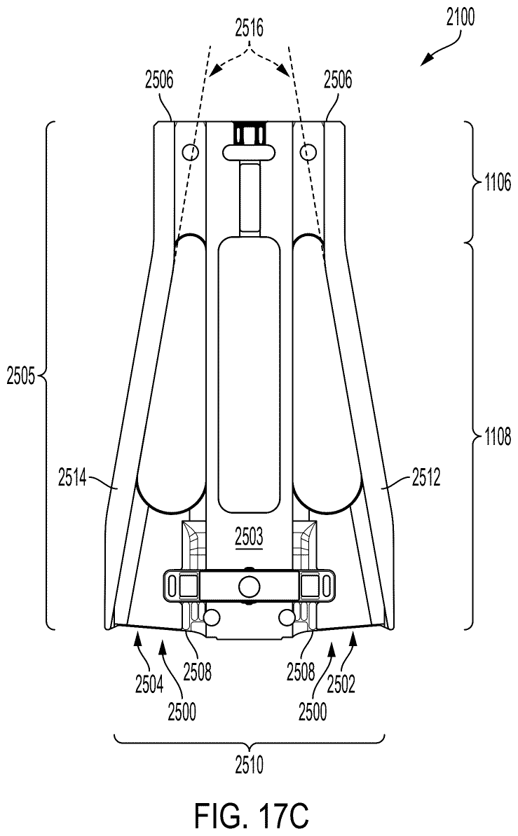

FIGS. 17A-17C (collectively “FIG. 17”) are different views of an example configuration of staple implant insertion device 2100 in which the staple implant insertion device includes a converging receiving cavity configured to receive a first coupling shaft 1102 and a second coupling shaft 1104 connected to a first handling coupling and a second handling coupling, respectively, of staple 2000 (or other staple configuration, as described herein). FIG. 17A is a perspective view of staple implant insertion device 2100. FIG. 17B is a side view of staple implant insertion device 2100 from FIG. 17A. FIG. 17C is a side sectional view of staple implant insertion device 2100 from FIG. 17B taken along the AA sectional line indicated on FIG. 17B.

In the illustrated example of FIG. 17, staple implant insertion device 2100 defines previously described connector 1106 and guide body 2102. In particular, as illustrated, guide body 2102 extends longitudinally from connector 1106 to form a monolithic or unitary instrument, with guide body 2102 being configured to guide insertion of first coupling shaft 1102 and second coupling shaft 1104 to connector 1106. Staple implant insertion device 2100 in FIG. 17 also includes a previously-described first guide lumen 2108 and second guide lumen 2110, with the number and arrangement of guide lumens varying depending on the number and arrangement of fixation apertures of the staple being inserted using the device.

Staple implant insertion device 2100 in the example of FIG. 17 can define a receiving space into which the proximal ends first coupling shaft 1102 and a second coupling shaft 1104 can be inserted, when the coupling shafts are attached to a staple and the staple is in its unenergized or undeformed state, resulting in the proximal ends of the coupling shafts being spaced apart from each other a maximum distance (corresponding to the undeformed shape of the staple). The receiving space defined by staple implant insertion device 2100 can define a converging pocket that causes the proximal ends of first coupling shaft 1102 and a second coupling shaft 1104 to push towards each other as the proximal ends of the coupling shafts are advanced farther into the receiving space to connector 1106.

In use, receiving space defined by staple implant insertion device 2100 to allow the proximal ends of first coupling shaft 1102 and second coupling shaft 1104 to enter the receiving space without requiring the user to pre-compress the coupling shafts together. As the user continues to advance staple implant insertion device 2100 down over the two coupling shafts, the receiving space can narrow or converge, causing the first and second coupling shafts to push or compress toward each other. The receiving space can be sized such that, when first coupling shaft 1102 and second coupling shaft 1104 are inserted to a maximum depth allowed by the device, the bridge of the staple is deformed so the legs of the staple attached to the coupling shafts are parallel to each other for insertion into corresponding bone portions.

With reference to FIG. 17C, guide body 2102 extends lengthwise from connector 1106 to form device 2100. Device 2100 can define at least one receiving opening 2500 configured to receive a proximal portion of first coupling shaft 1102 and a proximal portion of second coupling shaft 1104 inserted therein. In the illustrated configuration, the at least one receiving opening 2500 includes a first receiving opening 2502 configured to receive first coupling shaft 1102 therein and a second receiving opening 2504 configured to receive second coupling shaft 1104 therein. The first receiving opening 2502 and second receiving opening 2504 are separated by a divider 2503 centered axially along a length of device 2100.

The at least one receiving opening 2500 can define a length 2505 extending from a first lengthwise end 2506 to a second lengthwise end 2508, where the second lengthwise end is configured to be positioned closer to the staple than the first lengthwise end. The at least one receiving opening 2500 can also define a width 2510 extending perpendicular to the length. As seen in FIG. 17, width 2510 of receiving opening 2500 (e.g., each first receiving opening 2502 and second receiving opening 2504) can reduce or narrow from second lengthwise end 2508 to first lengthwise end 2506. This can result in the separation distance between first coupling shaft 1102 and second coupling shaft 1104 reducing as the proximal end of each coupling shaft is advanced progressively farther into the receiving opening from second lengthwise end 2508 toward first lengthwise end 2506.

In the example configurations of staple implant insertion device 2100 in FIGS. 11-15, first coupling shaft 1102 and second coupling shaft 1104 are shown as positioned external to wall surfaces defining guide body 2102 until reaching bounded receiving openings defined by connector 1106. By contrast, in the example of FIG. 17, first coupling shaft 1102 and second coupling shaft 1104 are configured to be received internal to external wall surfaces defined by guide body 2102.

For example, with further reference to FIG. 17C, staple implant insertion device 2100 is shown as defining a first side wall 2512 and a second side wall 2514. The total width 2510 of receiving opening 2500 extends from an internal surface of first side wall 2512 to an internal surface of second side wall 2514. As shown divided into two receiving openings by divider 2503, first receiving opening 2502 extends from the internal surface of first side wall 2512 to divider 2503, and second receiving opening 2504 extends from the internal surface of second side wall 2514 to divider 2503.

The width 2510 of receiving opening 2500 can narrow from second lengthwise end 2508 to first lengthwise end 2506. The receiving opening 2500 can narrow at an angle 2516 measured between a sloped portion of the internal surface of first side wall 2512 and a sloped portion of the internal surface of second side wall 2514. In some configurations, the internal surfaces of first side wall 2512 and second side wall 2514 slope continuously over their entire length from second lengthwise end 2506 to first lengthwise end 2506. In other examples, first side wall 2512 and second side wall 2514 slope over only a portion of their length and/or the angle of slope varies for one or both side walls over their length. For example, the internal surface of first side wall 2512 and second side wall 2514 define an orthogonal (non-sloping) region adjacent first lengthwise end 2506 that transitions to a sloping region extending to second lengthwise end 2508. Angle 2516 can be measured at a location of maximum slope between the internal surfaces of first side wall 2512 and second side wall 2514. In some examples, angle 2516 is with a range from 25 degrees to 80 degrees, such as from 30 degrees to 60 degrees.

In general, the separation distance between the internal surfaces of first side wall 2512 and second side wall 2514 may narrow from second lengthwise end 2508 to first lengthwise end 2506 (e.g., as defined by angle 2516) an amount effective cause first coupling shaft 1102 and second coupling shaft 1104 to move from a position in which staple to which the shafts are connected is in an undeformed state upon insertion of the first coupling shaft and the second coupling shaft at second lengthwise end 2508 to a position in which the first coupling shaft and the second coupling shaft apply a load force at the staple causing first leg 2008 and second leg 2010 to move apart from one another (in addition to causing third leg 2016 and forth leg 2018 to move apart from one another, which so configured), when the proximal portion of the first coupling shaft and the proximal portion of the second coupling shaft are advanced toward first lengthwise end 2506.

For example, the separation distance between the internal surfaces of first side wall 2512 and second side wall 2514 may narrow from second lengthwise end 2508 to first lengthwise end 2506 (e.g., as defined by angle 2516) an amount effective cause first coupling shaft 1102 and second coupling shaft 1104 to elastically deform staple 2000 until bridge 2206 (e.g., a top surface of the bridge) to be substantially planar, when the proximal end of the first coupling shaft and the proximal end of the second coupling shaft are advanced to a maximum extent allowed by the device toward first lengthwise end 2506 (e.g., where the maximum extent allowed by the device corresponds to the intended depth of insertion when the coupling shafts are fully inserted).

Additionally or alternatively, the separation distance between the internal surfaces of first side wall 2512 and second side wall 2514 may narrow from second lengthwise end 2508 to first lengthwise end 2506 (e.g., as defined by angle 2516) an amount effective cause first coupling shaft 1102 and second coupling shaft 1104 to elastically deform staple 2000 until first leg 2008 and second leg 2010 are parallel to each other (e.g., in addition to causing third leg 2016 and forth leg 2018 to be parallel to each other, which so configured), when the proximal end of the first coupling shaft and the proximal end of the second coupling shaft are advanced to a maximum extent allowed by the device toward first lengthwise end 2506.

In some examples, the separation distance between the internal surfaces of first side wall 2512 and second side wall 2514 may narrow from second lengthwise end 2508 to first lengthwise end 2506 (e.g., as defined by angle 2516) an amount effective cause first coupling shaft 1102 and second coupling shaft 1104 to move from a position in which the proximal ends of coupling shafts are angled away from each other to a position in which the proximal ends of coupling shafts are parallel to each other, when the proximal end of the first coupling shaft and the proximal end of the second coupling shaft are advanced to a maximum extent allowed by the device toward first lengthwise end 2506.

In other examples, the separation distance between the internal surfaces of first side wall 2512 and second side wall 2514 may narrow from second lengthwise end 2508 to first lengthwise end 2506 (e.g., as defined by angle 2516) an amount effective cause first coupling shaft 1102 and second coupling shaft 1104 to move from a position in which the proximal ends of coupling shafts are angled away from each other to a position in which the proximal ends of coupling shafts are angled toward each other, when the proximal end of the first coupling shaft and the proximal end of the second coupling shaft are advanced to a maximum extent allowed by the device toward first lengthwise end 2506. Such over compression or biasing of the coupling shafts toward each other may be used if needed to apply sufficient force to the connected staple to move the legs of the staple into parallel alignment with each other.

In some examples, a center of first receiving opening 2502 is separated from a center of second receiving opening 2504 at first lengthwise end 2506 by a first separation distance (measured widthwise across device 2100). The center of first receiving opening 2502 can also be separated from the center of second receiving opening 2504 at second lengthwise end 2508 a second separation distance (also measured widthwise across device 2100). In some examples, the second separation distance is greater than the first separation distance an amount within a range from 5 mm to 25 mm, such as from 5 mm to 10 mm, from 5 mm to 25 mm, from 10 mm to 35 mm, or from 10 mm to 25 mm. The proximal ends of the coupling shafts may move toward each other a distance within the foregoing ranges upon being inserted into staple implant insertion device 2100.

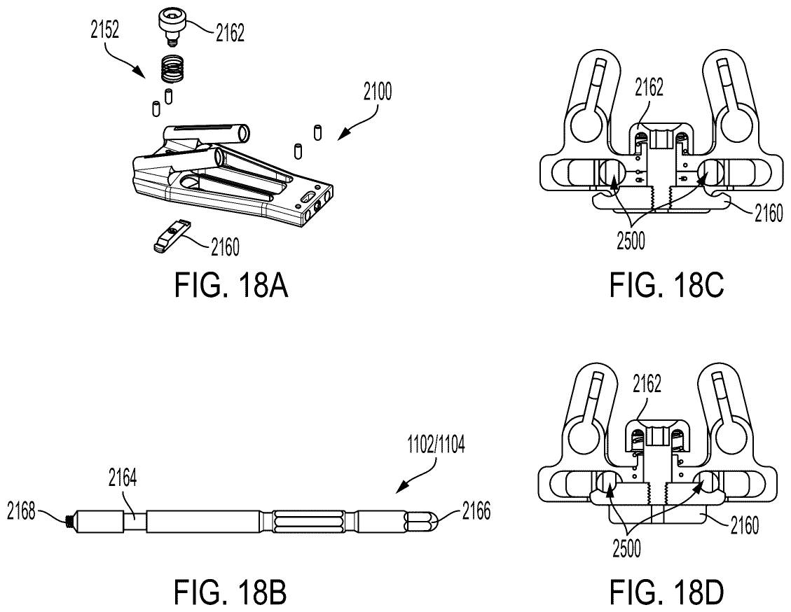

As previously discussed, staple implant insertion device 2100 can include a lock 2152 that is configured to lock connector 1106 and/or guide body 2102 to first coupling shaft 1102 and/or second coupling shaft 1104. This can prevent staple implant insertion device 2100 from inadvertently separating from one or both coupling shaft, e.g., when drilling through first guide lumen 2108 and/or second guide lumen 2110. FIGS. 18A-18D illustrate different example lock features that can be used on staple implant insertion device 2100 according to the disclosure. FIG. 18A is an exploded perspective view of an example configuration of the lock. FIG. 18B is a side view of an example configuration of a coupling shaft that can engage with the lock. FIGS. 18C and 18D are bottom views of staple implant insertion device 2100 showing example lock positions in an open and closed position, respectively.

With reference to FIG. 18A, lock 2152 may include an engagement body 2160 that is connected to a biasing member that is configured to biased engagement body in contact with first coupling shaft 1102 and/or second coupling shaft 1104, when inserted into staple implant insertion device 2100. Engagement body 2160 can form a movable projection that can engage with a corresponding recess of first coupling shaft 1102 and/or second coupling shaft 1104. Lock 2152 may include an actuator (e.g., button) 2162 that a user can engage to disengage lock 2152 from first coupling shaft 1102 and/or second coupling shaft 1104.

With reference to FIG. 18B, first coupling shaft 1102 and/or second coupling shaft 1104 may include a recessed region 2164 extending partially or fully about a perimeter of the coupling shaft. Recessed region 2164 may be located at a lengthwise location between a proximal end 2166 of the coupling shaft in a distal end 2168 of the coupling shaft (which is configured to couple to the staple thereat). The lengthwise location where recessed region 2164 is located may be selected such that engagement body 2160 engages the recessed region when the coupling shaft is inserted into staple implant insertion device 2100 to a desired depth (e.g., a maximum extent). Depending on the configuration of the locking arrangement, in some applications, staple implant insertion device 2100 may provide an audible output to the user when first coupling shaft 1102 and/or second coupling shaft 1104 is inserted into staple implant insertion device 2100 to a desired depth (e.g., a maximum extent). The audible output may be a click or other mechanical sound associated with engagement of lock 2152 with first coupling shaft 1102 and/or second coupling shaft 1104.

FIG. 18C illustrates actuator 2162 engaged to cause engagement body 2160 to move widthwise out of alignment with the at least one receiving opening 2500 of staple implant insertion device 2100 and/or out of recessed region 2164 of the coupling shaft, when inserted therein. FIG. 18D illustrates actuator 2162 released such that the biasing member causes engagement body 2160 to move widthwise into the at least one receiving opening 2500 of staple implant insertion device 2100 and/or into recessed region 2164 of the coupling shaft, when inserted therein.

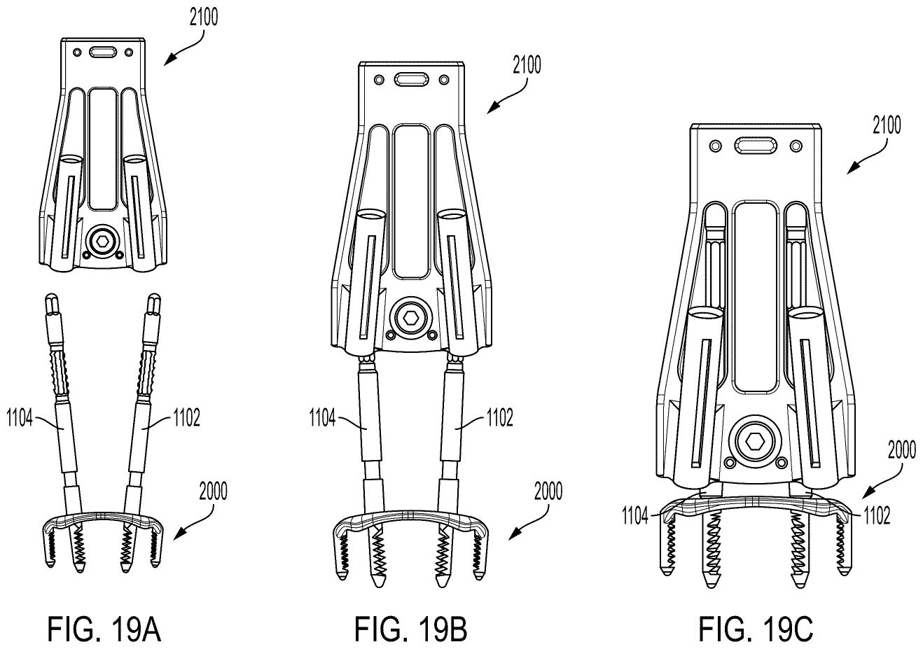

FIGS. 19A-19C are side view illustrations of an example configuration of staple implant insertion device 2100 showing example procedure steps for engaging the staple implant insertion device with first coupling shaft 1102 and second coupling shaft 1104 attached to a staple 2000. With reference to FIG. 19A, first coupling shaft 1102 and second coupling shaft 1104 can be attached to staple 2000 with the staple in a undeformed state, resulting in the proximal end of first coupling shaft 1102 being angled away from the proximal end of second coupling shaft 1104. With reference to FIG. 19B, a user can position the at least one receiving opening 2500 of staple implant insertion device 2100 over the proximal ends of the two coupling shafts and move the components relative to each other to cause the proximal end of the first coupling shaft 1102 and proximal end of the second coupling shaft 1104 to be initially introduced into the receiving opening. With reference to FIG. 19C, the user can advance the proximal end of first coupling shaft 1102 and the proximal end of second coupling shaft 1104 farther into the at least one receiving opening 2500 (e.g., until lock 2152 engages with one or both coupling shafts), thereby causing the first coupling shaft and the second coupling shaft to move toward each other and, in turn, causing the distal end of the legs of the staple to move apart from one another (e.g., moving the legs into parallel alignment with each other).

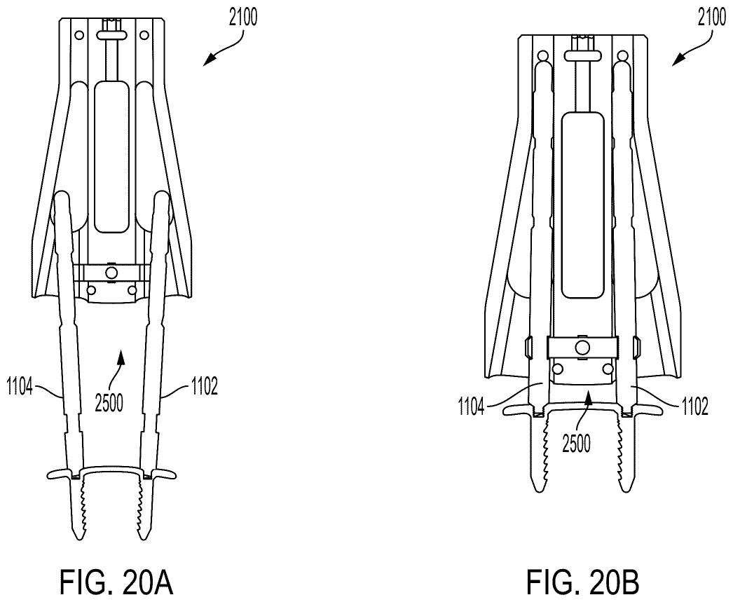

FIGS. 20A and 20B are sectional side views of an example configuration of staple implant insertion device 2100 showing first coupling shaft 1102 and second coupling shaft 1104 attached to a staple and being inserted into an example receiving opening of the device. FIG. 20A illustrates how the proximal ends of the coupling shafts contact the internal walls surfaces of the device as the internal walls surfaces converge inwardly, causing the shafts to move toward each other. FIG. 20B illustrates first coupling shaft 1102 and second coupling shaft 1104 fully inserted into staple implant insertion device 2100 with the coupling shafts in parallel alignment to each other and the legs of the staple attached thereto also in parallel alignment.

In use, a clinician can prepare staple leg openings by drilling holes through corresponding staple leg guide openings of staple leg drill guide. The clinician can attach first coupling shaft 1102 and second coupling shaft 1104 to a staple (if not already pre-installed by a manufacturer or other provider) and insert the coupling shafts into staple implant insertion device 2100. As first coupling shaft 1102 and second coupling shaft 1104 are inserted into staple implant insertion device 2100, the proximal ends of the coupling shafts can be forced toward each other due to the converting internal walls of the device, causing the staple to transition from an undeformed state to a deformed state. In some examples, the clinician inserts the coupling shafts into staple implant insertion device 2100 until a lock engages one both coupling shafts, which may be indicated by an audible mechanical clicking sounds. In either case, the clinician can manipulate staple implant insertion device 2100 to position the legs of the staple into the holes formed in the bone portions.

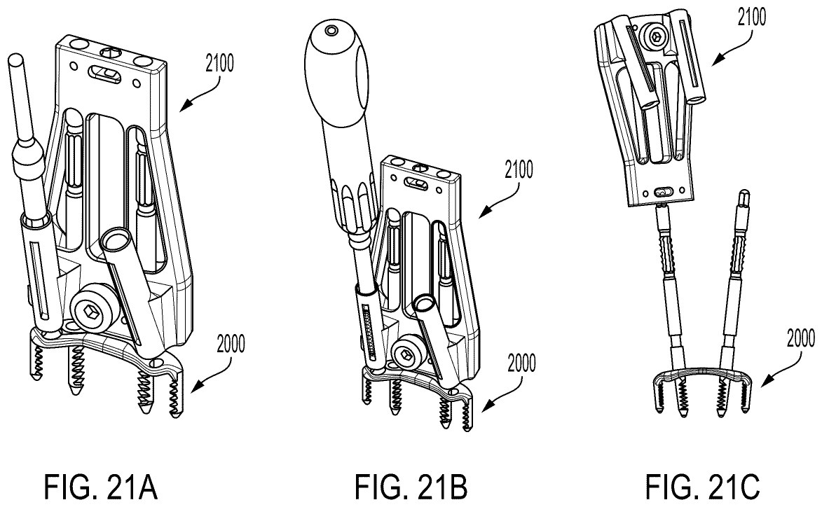

FIGS. 21A-21C are perspective views of example procedural steps that can be performed with staple implant insertion device 2100 after inserting the legs of the staple into bone portions. With reference to FIG. 21A, the clinician can insert a drill bit or other hole forming instrument through each guide opening defined by staple implant insertion device 2100 and through each fixation aperture of the staple aligned therewith to form a hole in each bone portion aligned with each fixation aperture. With reference to FIG. 21B, the clinician can withdraw the drill bit or other hole forming instrument through each guide opening and insert a fixation element (e.g., screw attached to a screw driver) through each guide opening. The fixation element can through each fixation aperture of the staple and hole aligned therewith until the fixation is secured at and/or in the fixation aperture of the staple. Thereafter, the clinician remove staple implant insertion device 2100 from first coupling shaft 1102 and second coupling shaft 1104, e.g., by pulling the device off the coupling shafts and thereby releasing the force pushing the shafts away from each other by the device.

With reference to FIG. 21C, in some configurations, staple implant insertion device 2100 may include a built-in driver connection (e.g., a hex connection, a hexalobe connection, a cruciform connection, a slotted connection) configured to engage the proximal end of first coupling shaft 1102 and second coupling shaft 1104. The clinician can use the driver connection to engage the proximal end of first coupling shaft 1102 and second coupling shaft 1104 to detach the coupling shafts from the staple (e.g., by rotationally disconnecting each shaft from the staple).

Various examples have been described. These and other examples are within the scope of the following claims.

1. An implant insertion system comprising:
a staple comprising a first leg and a first fixation aperture on a first side of the staple, a second leg and a second fixation aperture on a second side of the staple, a bridge connecting the first side of the staple to the second side of the staple, a first handling coupling extending through a top surface of the staple on the first side of the staple, and a second handling coupling extending through the top surface of the staple on the second side of the staple;
a first coupling shaft configured to connect to the first handling coupling through the top surface of the staple;
a second coupling shaft configured to connect to the second handling coupling through the top surface of the staple;
a connector that is configured to join the first coupling shaft and the second coupling shaft, wherein, when the first coupling shaft is connected to the first handling coupling, the second coupling shaft is connected to the second handling coupling, and the connector joins the first coupling shaft and the second coupling shaft, the first coupling shaft and the second coupling shaft are configured to apply a load force at the staple to cause the first leg and the second leg to move apart from one another; and
a guide body operatively connected to the connector that defines one or both of:
a first guide opening configured to guide a drill bit for drilling a first hole into a first bone aligned with the first fixation aperture and/or insertion of a first screw into the first bone through the first fixation aperture; and
a second guide opening configured to guide the drill bit for drilling a second hole into a second bone aligned with the second fixation aperture and/or insertion of a second screw into the second bone through the second fixation aperture.

2. The system of claim 1, wherein the guide body defines both the first guide opening and the second guide opening.

3. The system of claim 1, wherein:
the guide body has a first guide lumen defining the first guide opening, the first guide lumen extending from a first end to a second end, the second end being positioned closer to the staple than the first end; and/or
the guide body has a second guide lumen defining the second guide opening, the second guide lumen extending from a first end to a second end, the second end being positioned closer to the staple than the first end.

4. The system of claim 3, wherein:
the guide body extends parallel to the first coupling shaft and the second coupling shaft to define a substantially planar face; and
the first guide lumen and/or second guide lumen extend angularly outwardly from the substantially planar face.

5. The system of claim 3, further comprising:
a first guide tube at least partially insertable into the first guide lumen; and/or
a second guide tube at least partially insertable into the second guide lumen.

6. The system of claim 1, further comprising a staple leg drill guide comprising a first staple leg guide opening configured to guide a hole forming instrument to form a first staple leg hole into the first bone and a second staple leg guide opening configured to guide the hole forming instrument to form a second staple leg hole into the second bone, the first staple leg hole and the second staple leg hole being configured to be positioned at a location for receiving the first leg and the second leg of the staple, respectively.

7. The system of claim 1, wherein:
the connector comprises a first receptacle and a second receptacle, the first coupling shaft is received in the first receptacle, and the second coupling shaft is received in the second receptacle; and
the guide body extends from the connector toward the staple between the first coupling shaft and the second coupling shaft.

8. The system of claim 1, wherein the guide body is integrally formed with the connector.

9. The system of claim 1, wherein:
the guide body extends lengthwise from the connector to form an insertion device defining at least one receiving opening configured to receive a proximal portion of the first coupling shaft and a proximal portion of the second coupling shaft inserted therein;
the at least one receiving opening has a length and a width;
wherein the length of the at least one receiving opening extends from a first lengthwise end to a second lengthwise end, the second lengthwise end being configured to be positioned closer to the staple than the first lengthwise end; and
the width of the at least one receiving opening narrows from the second lengthwise end to the first lengthwise end.

10. The system of claim 9, wherein:
the at least one receiving opening comprises a first receiving opening configured to receive the proximal portion of the first coupling shaft inserted therein and a second receiving opening configured to receive the proximal portion of the second coupling shaft inserted therein; and
the first receiving opening is separated from the second receiving opening by a divider extending along at least a portion of the length of the insertion device.

11. The system of claim 10, wherein:
insertion device comprises a first side wall and a second side wall;
the width extends from an internal surface of the first side wall to an internal surface of the second side wall;
the first receiving opening extends from the internal surface of the first side wall to the divider; and
the second receiving opening extends from the internal surface of the second side wall to the divider.

12. The system of claim 9, wherein the width of the at least one receiving opening narrows from the second lengthwise end to the first lengthwise end at an angle configured to cause the first coupling shaft and the second coupling shaft, when connected to the staple, to move from a position in which the staple is in an undeformed state upon insertion of the first coupling shaft and the second coupling shaft at the second lengthwise end to a position in which the first coupling shaft and the second coupling shaft apply the load force at the staple causing the first leg and the second leg to move apart from one another, when the proximal portion of the first coupling shaft and the proximal portion of the second coupling shaft are advanced toward the first lengthwise end.

13. The system of claim 12, wherein the angle is configured to cause the first leg of the staple to be substantially parallel to the second leg of the staple, when the proximal portion of the first coupling shaft and the proximal portion of the second coupling shaft are advanced to a maximum extent toward the first lengthwise end.

14. The system of claim 1, further comprising a lock configured to releasably lock the first coupling shaft and the second coupling shaft to the connector and/or the guide body.

15. The system of claim 14, wherein:
the first coupling shaft and the second coupling shaft each include a recessed region; and
the lock comprises a movable projection insertable into the recess region of the first coupling shaft and the second coupling shaft.

16. The system of claim 1, wherein:
the staple further comprises a third leg on the first side of the staple and a fourth leg on the second side of the staple; and
the first fixation aperture if positioned between the first leg and the third leg, and the second fixation aperture is positioned between the second leg and the fourth leg.

17. A method comprising:
guiding a drill bit through a first staple leg guide opening of a staple leg drill guide to form a first staple leg hole in a first bone;
guiding the drill bit through a second staple leg guide opening of the staple leg drill guide to form a second staple leg hole in a second bone;
with first and second coupling shafts attached to a staple and joined together via a connector to cause a first leg and a second leg of the staple to move apart from one another, inserting the first leg of the staple into the first staple leg hole and the second leg of the staple into the second staple leg hole;
guiding the drill bit through a first guide opening of a guide body operatively connected to the connector to form a first hole in the first bone aligned with a first fixation aperture of the staple and subsequently guiding a first screw through the first guide opening through the first fixation aperture and into the first hole; and
guiding the drill bit through a second guide opening of the guide body operatively connected to the connector to form a second hole in the second bone aligned with a second fixation aperture of the staple and subsequently guiding a second screw through the second guide opening through the second fixation aperture and into the second hole.

18. The method of claim 17, wherein:
guiding the drill bit through the first guide opening of the guide body operatively connected to the connector to form the first hole comprises guiding the drill bit through a first guide tube aligned with and/or at least partially inserted into the first guide opening and subsequently removing the first guide tube before guiding the first screw through the first guide opening through the first fixation aperture and into the first hole; and
guiding the drill bit through the second guide opening of the guide body operatively connected to the connector to form the second hole comprises guiding the drill bit through a second guide tube aligned with and/or at least partially inserted into the second guide opening and subsequently removing the second guide tube before guiding the second screw through the second guide opening through the second fixation aperture and into the second hole.

19. The method of claim 17, wherein the first bone is a metatarsal and the second bone is a phalanx.

20. The method of claim 17, wherein:
the guide body extends lengthwise from the connector to form an insertion device defining at least one receiving opening configured to receive a proximal portion of the first coupling shaft and a proximal portion of the second coupling shaft inserted therein;
the proximal portion of the first coupling shaft and the proximal portion of the second coupling shaft are inserted into the at least one receiving opening while inserting the first leg of the staple into the first staple leg hole and the second leg of the staple into the second staple leg hole; and
the proximal portion of the first coupling shaft and the proximal portion of the second coupling shaft are inserted into the at least one receiving opening while guiding the drill bit through the first guide opening of the guide body, guiding the first screw through the first guide opening, guiding the drill bit through the second guide opening of the guide body, and guiding the second screw through the second guide opening.

21. The method of claim 20, further comprising, with first and second coupling shafts attached to the staple:
positioning a proximal end of the first coupling shaft and a proximal end of the second coupling shaft in the at least one receiving opening with the staple in an undeformed state; and
advancing the proximal end of the first coupling shaft and the proximal end of the second coupling shaft farther into the at least one receiving opening, thereby causing the first coupling shaft and the second coupling shaft to move toward each other and, in turn, causing the first leg and the second leg to move apart from one another.

22. The method of claim 20, wherein advancing the proximal end of the first coupling shaft and the proximal end of the second coupling shaft farther into the at least one receiving opening comprises advancing the proximal end of the first coupling shaft and the proximal end of the second coupling shaft to a maximum extent into the at least one receiving opening, causing a top surface of a bridge of the staple to be substantially planar.

23. A staple implant insertion device comprising:
a connector that is configured to join a first coupling shaft couplable to a staple and a second coupling shaft couplable to the staple; and
a guide body operatively connected to the connector that defines:
a first guide opening configured to guide a drill bit for drilling a first hole into a first bone aligned with a first fixation aperture of the staple and/or insertion of a first screw into the first bone through the first fixation aperture, when the first coupling shaft and the second coupling shaft are coupled to the staple and joined by the connector; and
a second guide opening configured to guide the drill bit for drilling a second hole into a second bone aligned with a second fixation aperture of the staple and/or insertion of a second screw into the second bone through the second fixation aperture, when the first coupling shaft and the second coupling shaft are coupled to the staple and joined by the connector.
